(19) United States
(12) Patent Application Publication
(10) Pub. No.: US 2022/0285026 A1
(43) Pub. Date: Sep. 8, 2022

(54) Applying non-real time and non-user attended algorithms to the stored non-imaging data and existing imaging data to obtain a dental diagnosis

(71) Applicants: Douglas A. Golay, Coon Rapids, IA (US); Wyatt C. Davis, Bozeman, MT (US)

(72) Inventors: Douglas A. Golay, Coon Rapids, IA (US); Wyatt C. Davis, Bozeman, MT (US)

(21) Appl. No.: 17/700,379

(22) Filed: Mar. 21, 2022

Related U.S. Application Data
(63) Continuation-in-part of application No. 16/537,709, filed on Aug. 12, 2019.
Application No. 16/537,709 is a continuation-in-part of application No. 14/456,255, filed on Aug. 11, 2014, now abandoned.

(51) Int. Cl. G16H 50/20; G16H 10/60; A61B 6/14; A61C 19/04

(52) U.S. Cl. CPC G16H 50/20; G16H 10/60; A61B 6/14; A61C 19/04; G06T 2207/30036

(57) ABSTRACT

The present invention is a method of making a diagnosis of a dental condition of a patient which includes the steps of collecting non-imaging data relating to the patient, storing the non-imaging data in a storage medium containing stored non-imaging data and existing imaging data for this patient and for a plurality of other patients and applying non-real time and non-user attended algorithms to the stored non-imaging data and the existing imaging data of this patient and other patients. The algorithms determine the diagnosis of the dental condition of the patient. The diagnosis either is complete or determines what new dental imaging data for the patient is required to be acquired to diagnose the dental condition of the patient. The non-imaging data includes non-clinical data and non-dental clinical data.

### BACKGROUND OF THE INVENTION

### Field of the Invention

The present invention relates to a method of making a diagnosis of a dental condition of a patient includes the steps of collecting non-imaging data relating to the patient, storing the non-imaging data in a storage medium containing stored non-imaging data and existing imaging data for this patient and for a plurality of other patients and more particularly applying non-real time and non-user attended algorithms to the stored non-imaging data and existing imaging data in order to obtain a dental diagnosis.

### Description of the Prior Art

In the field of dentistry, dentists routinely use intra-oral, extra-oral, and 3D x-rays to visually inspect patient's teeth for dental conditions such as caries, fractures, bone loss, and orthodontic procedures. The dentist uses these x-rays and other clinical aides such as an explorer and visual inspection to decide if any treatment is required and if so whether the condition requires immediate treatment or increased preventative care. Dentists also use various forms of color or video images of teeth to detect bacteria levels, trans-illumination for showing and detection of cracks, and photographic images for cosmetic documentation and simulations. As preventative and diagnostic dentistry techniques and the physical number of dental imaging devices continue to advance it is becoming increasingly difficult for dentists to properly screen all the above types of images and for all the various conditions in real-time or semi real-time when utilizing the time available during an appointment and/or during office hours. Likewise, there are many various technologies available for diagnostic and preventative procedures and most dentists do not have all the various products and technologies available in the practice for routine use and even if they did there would not exist enough time in a standard patient appointment visit to apply all of the available techniques and technologies. Another issue is that most dentists have disparate imaging equipment from multiple manufacturers of 2D imaging and 3D imaging systems which do not directly integrate or share images such as is often the case in the medical world with Dicom/PACS types of systems. Imaging software is not usually provided by the Practice Management/EMR software vendor in a typical dentist's office and are 3rd party vendors imaging software. When bridges exist between practice management software and Dicom/PACS systems or 3rd party imaging systems these systems are often too complicated for the general dentist to deploy and maintain and are still neither 100% bi-directionally integrated nor capable of sharing all image data and original image and non-image related patient information. The above disparate imaging systems prevent useful data mining of dental practice management records simultaneously with automated image data analysis for detection of specific dental conditions. Having locally installed disparate equipment and imaging software's which save images and data locally in the dental office make it nearly impossible to use multiple image types such as intraoral, extraoral, or cone beam images from multiple imaging devices and or using multiple non-affiliated dental practices in the analysis for detection of specific dental conditions.

U.S. Pat. No. 9,675,305 teaches a system for determining an orthodontic diagnostic analysis of a patient at various dental maturity stages which predicts future conditions and/or treatment recommendations. The system locates points in a mouth of a patient using an imaging device wherein the imaging device generates imaging data. The imaging data is transferred to a central processing unit wherein the central processing unit has access to a database having information associated with orthodontic conditions stored therein. The central processing unit obtains measurements associated with selected points and dentition in the mouth of the patient and predicts orthodontic conditions of the patient based upon the measurements and the information in the database. The central processing unit recommends treatments to the patient based upon the predicted orthodontic conditions.

It is generally known to provide dental care to a patient. The patient may seek care from a professional at an office visit. The professional may be a dentist, an orthodontist, or other type of oral health care provider. The professional may examine the patient using various techniques. Such techniques may be imaging and/or x-raying the oral area and/or the jaws. After reaching a diagnosis, the professional may then provide the patient with an oral appliance to correct the condition of the patient. In addition to the oral appliance, the professional may provide the patient with instructions for exercises to perform while wearing the oral appliance. The exercises may cause the teeth to move toward a corrected position and may assist in correcting a malocclusion. Diagnostic decisions may often be made by a single look at the patient by the professional. The professional may estimate what may be present in the dentition of the patient. The examination may not entail a deeper and/or more detailed study. The thoroughness of the examination may seriously impact the future of the patient. The individual deciding the best alternative for a patient may have little understanding of how future development of the various problems may influence the outcome of the future health of a patient. Several analytical procedures that may be significant may seldom be used to make a diagnosis for a patient. The patient may ultimately suffer as a result. A typical example may be an arch-length analysis. The arch-length measurement may accurately predict if sufficient room may be available to straighten crowded teeth and/or rotated teeth. The arch-length analysis may be time consuming for the professional. As a result, some arch-length analyses may provide an inaccurate assessment. Another important consideration in the assessment of the dental health of the patient may be the age of the patient. Dental maturity may be generally categorized into five age groups of which four groups may be segregated according to dental maturity stages. The four stages may be the full deciduous dentition from about two years of age or three years of age up to about five and one-half years of age or six years of age. The permanent lower incisors may begin to erupt at about five and one-half years of age to six and one-half years of age. The period during which the adult incisors may begin and finish their full eruption may be at about seven years of age or eight years of age may be called the transitional period. The next dental maturity stage may be called the mixed-dentition period when the other permanent teeth, such as canines, first premolars, second premolars and the permanent second molars may erupt into place. This period may last from about eight years of age to twelve years of age. The next dental maturity stage may be the adult dentition where twenty-eight permanent teeth may be fully erupted and where jaw growth may still be active up to about eighteen years of age in a female and about twenty years of age in a male. The final dental maturity stage may be during the adult dentition after most of the jaw growth may be complete. Although both males and females grow slightly after this period, this minimal growth is not generally important for orthodontic treatment. Most orthodontics may be done during the late mixed stage and the early adult dentition from about eleven years of age to thirteen years of age. Some orthodontics may be done during the mixed dentition after the permanent upper and lower permanent incisors may be erupted. Orthodontics are infrequently used before or during the eruption of the adult incisors. Performing orthodontics during the transitional eruption period may have the advantage that the teeth may be aligned before the collagenous fibers may be formed. The orthodontics may minimize relapse tendencies and may lessen the length of treatment to about twenty percent of the average time consumed for fixed orthodontics for patients of eleven years of age to thirteen years of age. Treatment with fixed and/or removable appliances during the transitional period on patients of six years of age to eight years of age and earlier on patients of two years of age to six years of age may be beneficial in malocclusion treatment. The early period with patients of two years of age to six years of age may be recommended for sleep-disordered breathing problems. The treatment may either advance the mandible and tongue or may prevent the lower jaw from displacing posteriorly while sleeping. The treatment may teach the patient to breathe through the nose instead of the mouth which may correct the snoring and may improve the behavioral symptoms caused by breathing problems. Child patients that may have a prominent mandible may be helped at a young age by treatment to slow adverse changes that may occur during the growing years. Further types of correction that may improve breathing may entail improving abnormal swallowing, correcting anterior open bites, correcting a narrowed maxilla, and improving speech problems. Such early problems may have significant effects on the future health and well-being of the patient. In general dentistry, oral surgery, maxillofacial surgery and/or orthodontics, malocclusions may be assessed clinically or radiographically using cephalometries. One such common condition of a malocclusion may be overbite, in which the top teeth and/or the lower teeth of the patient do not align properly. Cephalometric analysis may be the most accurate way of determining types of malocclusions, since such analysis may include assessments of skeletal body, occlusal plane angulation, facial height, soft tissue assessment and anterior dental angulation. Various calculations and assessments of the information in a cephalometric radiograph may allow the clinician to objectively determine dental relationships and/or skeletal relationships and determine a plan of correction. If a non-surgical alternative may produce results comparable with those that may be achieved surgically, then the professional may consider and/or may suggest such a non-surgical approach to the patient. In some cases, a non-surgical approach may be the preferred choice of the professional and/or the patient. Facial growth modification may be an effective method of resolving skeletal Class III jaw discrepancies in growing children. Dentofacial orthopedic appliances may be used. Orthognathic surgery in conjunction with orthodontic care may be required for the correction of malocclusions in an adult patient. A need, therefore, exists for a system and a method for determining an orthodontic diagnostic analysis of a patient at various dental maturity stages with predictions of future conditions and/or treatment recommendations. A need also exists for a system and a method that may use a computer for determining an orthodontic diagnostic analysis of a patient at various dental maturity stages with predictions of future conditions and/or treatment recommendations. A need also exists for a system and a method for determining an orthodontic diagnostic analysis of a patient at various dental maturity stages with predictions of future conditions and/or treatment recommendations that may use an oral appliance.

U.S. Patent Application Publication No. 2002/0029157 teaches a system which provides a computerized medical and biographical records database and diagnostic information. A medical records database and diagnostic program is stored on a central computer that is accessible to individuals using remotely situated computers connected to a computer network. Individual patient medical and biographical records are owned by individual patients who can enter information in their record as well as grant or deny authorization to others, such as health care professionals, insurance providers and other entities, to review part or all their record. The diagnostic program provides a series of diagnostic questions to an individual who must respond either “yes” or “no” to each question. Each potential response is weighted relative to its importance to a diagnosis of a particular disease. Relative weights for all responses to diagnostic questions are summed to identify potential diagnoses to connected to the answered questions. The diagnostic program provides the individual with a list of potential diagnoses as well as permitting the individual to save the information to his or her individual medical and biographical record. The information maintained in the above system and process is utilized for health care financing and insurance.

Patient medical and biographical records and medical diagnostic software are stored on a centralized computer accessible by remotely connected computers. The medical records are essentially “owned” by an individual patient who grants or denies varying degrees of access to the records to selected health care professionals based on the health care professional's field of specialty and need to know. The medical diagnostic software receives information provided by the patient and provides the patient with a list of potential medical diagnoses. This information also forms part of the patient's medical record. Medical record systems are well known in the prior art. Medical records have been used throughout the years of the practice of medicine in order to keep track of a patient's medical history, medical observations, diagnoses and any treatments prescribed to the patient. Often, a record contains information as to the success or failure of a particular treatment, a patient's allergies and reactions to drugs or treatments, and a record of patient visits. In addition to serving as a record of medical history and treatment, the medical record also serves as legal documentation of patient condition and treatment. Evolution of the health care system is engendering reevaluation of the roles of patients and health care providers regarding access and content of medical records. Long term relationships and trust between a family doctor and patient are no longer commonplace because a change in residence, job, or insurance carrier often requires the patient to change primary and/or specialty health care providers. Establishing relationships with a new health care provider can be tedious as medical records must first be transferred from previous health care providers and then reviewed by the new health care provider for past history, therapies, and present therapeutic regimes. Also, the new medical record being created by the new health care provider is often incomplete as patients frequently fail to remember to include all the necessary medical or biographical information. In fact, patients sometimes convey erroneous information that can be ultimately detrimental to their health. Control of the information contained in a patient's medical and biographical record is also becoming a significant public issue and a source of controversy and stress. Presently, such records are treated as being “owned” by the medical offices or institutions in which the records are housed. Distrust on maintenance of confidentiality results in failure to disclose information that may be important for health-care decisions. This distrust may be increased as patients transfer to new health care providers. Medical record systems usually consist of handwritten notes, pictures, and documents created by a medical and health care professional. Recently, computer programs and systems have become available for the generation, storage, and retrieval of medical records. In general, such systems operate on a computer owned by a hospital or other health care provider and may only be accessed by health care professionals that are affiliated with the health care provider. Patient medical information is typically input into a medical record by a physician, nurse, or other health care professional.

Several automated medical record systems have been designed and marketed in the health care field. U.S. Pat. No. 5,277,188 discloses a clinical information reporting system having an electronic database including electrocardiograph related patient data. Similarly, U.S. Pat. No. 5,099,424 discloses a computer system for recording electrocardiograph and/or chest x-ray test results for a database of patients. U.S. Pat. No. 4,315,309 discloses a patient report generating system for receiving, storing and reporting medical test data for a patient population. U.S. Pat. No. 3,872,448 likewise discloses a system for automatically handling and processing hospital data, such as patient information and pathological test information using a central processing apparatus. In U.S. Pat. No. 5,065,315, a computerized scheduling and reporting system is disclosed for managing information pertinent to a patient's stay in the hospital. Also, U.S. Pat. No. 5,924,074 discloses an electronic data processing system. While present automated systems may provide electronic storage of medical data, they typically suffer from significant shortcomings that have plagued medical record systems since their inception. These systems, like their paper record counterparts, are typically only available to health care professionals affiliated with the hospital, clinic, or other health care provider that owns the medical record software program and computer system. The information contained in a patient's medical record would not be able to be reviewed by another health care professional who is not affiliated with the health care provider that maintains the medical record software. This becomes an issue for patients who choose to be treated by a different health care provider or who may require treatment while traveling in a location not served by their usual health care provider. Treatment may be prescribed which has been previously determined to be ineffective or which is contraindicated for the patient.

Similarly, health care professionals from different health care providers may not be able to easily review a patient's medical record and confer with each other as to diagnosis and treatment. This may be due to either security controls by the health care provider or by incompatible systems used by different health care professionals. Medical professionals wishing to confer with each other may be required to copy and mail or send a facsimile of the patient's record, introducing privacy and control issues.

Since the existing systems are “owned” by the health care provider, a patient may be kept from reviewing his or her own medical record for the substance or accuracy of its information. Additionally, a patient cannot prevent, or control private information contained within the patient's medical record from being seen by any individual that has access to medical records, regardless of whether the individual has any right or need to review a particular portion of the patient's medical record. As such, information which the patient wishes to remain private may be reviewed, thereby compromising the patient's privacy and potentially introducing a negative bias to the health care professional towards the patient. An example of such information may include past treatment for a sexually transmitted disease or sexual dysfunction that may be irrelevant to a particular medical specialty. Current medical systems also often do not contain useful data such as family history, biographical data, genetic constitution or make-up, or other information that a patient may add to his or her medical record which could aid health care professionals in diagnosing the patient's condition or determine the best medical treatment.

Presently available medical records systems are not suited for providing medical diagnoses. Advancements in automation, research, specialization, and medical knowledge have permitted modern day health care to be increasingly improved over the care provided in the recent past. While these advancements have resulted in improved success rates of medical treatment, individuals often delay seeking medical attention due to fear of the unknown and the inconvenience of being referred to multiple physicians. Patient referrals typically occur when a primary care physician makes a general diagnosis, then refers a patient to a physician specializing in the diagnosis. Further referrals may occur if the patient is referred to medical sub-specialties for further diagnosis and treatment resulting in additional patient cost, time, and inconvenience. Patients who face these inconveniences and costs or who have experienced them in the past may delay seeking treatment in the hope that a condition may simply go away thereby precluding the need to seek the help of a health care professional. This delay can cause a medical condition which could be easily treated early in its development to require longer treatment, or the condition may even become untreatable by the time medical assistance is sought. If the same patients were informed of potential diagnoses of their conditions, they can be aware of the risks of delaying medical assistance and may be persuaded to seek help earlier. Informed patients may even be able to reduce the inconveniences of multiple referrals by initially seeking the assistance of a health care professional who specializes in treating their condition.

Medical information is readily attainable to the public through medical books available in libraries and bookstores, medical phone help or “Ask-A-Nurse” telephone services, audio visual informational programs on television and videotape, and Internet sites specializing in medical care. The amount of available information, however, can be overwhelming to an individual trying to determine the identification of his or her health condition who is unfamiliar with researching health information or who lacks a scientific background.

Computer programs have been developed to provide individuals potential diagnoses based on their responses to a series of health-related questions. U.S. Pat. Nos. 5,910,107 and 5,935,060 describe diagnostic programs which can be accessed over a telephone or computer network. An individual is asked a series of weighted questions concerning the individual's health symptoms and can respond with “yes,” “no,” or “not sure” answers or may be asked to answer multiple-choice questions. From the responses, the program identifies a list of potential diseases which are indicated by the individual's health symptoms. U.S. Pat. No. 5,572,421 discloses an electronic medical history questionnaire in which a patient can respond “yes,” “no,” or “not sure” to medical questions. The questionnaire then provides the physician with suggested tests that may be performed and conclusions regarding the patient's health. U.S. Pat. No. 5,839,438 discloses a diagnostic system using a neural network to provide a patient diagnosis to a physician from input data comprising measured and interview data regarding the patient's condition. The diagnosis is based upon a databases of physician diagnoses of medical conditions and their corresponding symptoms.

While prior art automated medical diagnostic programs diagnose a condition or confirm a diagnosis made by the physician, they are usually designed to be used by a physician and not a patient. The language and phrasing in these programs are designed for a medical professional and contain esoteric medical and health terms. Most patients do not understand these terms and cannot effectively use the programs. The diagnostic information provided by these programs does not inform individuals of their various conditions before they seek medical assistance. A further shortcoming of prior art automated diagnostic programs is that they can accept input data that is often erroneous or not helpful. As an individual may select “not sure” or other answers which are not simply “yes” or “no,” an individual is able to avoid answering conditions they feel are minor are irrelevant, but which may provide helpful data if the individual were forced to select only a “yes” or “no” response. A software program designed to accept objective data and provide individuals with diagnostic information about their health conditions would be desirable.

It would be beneficial to patients and health care professionals alike to develop an individual patient self-generated, fully controlled, and censored, centralized electronic medical and biographical records and medical diagnostic system that may be accessed by patients and health care professionals regardless of their affiliation with a particular hospital, clinic, or other health care provider. The medical and biographical records and medical diagnostic system would be maintained, stored, and delivered by a totally independent institution, not necessarily affiliated with the government, insurance, or health care industry. By using common language and phrasing tailored to different levels of education and familiarity with medical and health terms an individual could effectively utilize such a system to determine potential diagnoses prior to seeking medical attention, permit the individual to be better informed as to the potential medical specialty from which to seek assistance, and control the content of and access to the individual's medical record.

A self-generated record of present illness and pertinent information would also benefit individuals by allowing them ample opportunity to ponder and respond without encumbrances from health care providers presence. Such presence often generates discomfort or uneasiness and may lead to confused, unconsciously withheld, consciously suppressed information (e.g., suppressed for fear of embarrassment) or miscommunicated medical and biographical information.

A centralized electronic medical and biographical records and medical diagnostic system would also permit any health care professional to be aware of all a patient's biographical and medical history that is relevant to treating the patient. Additionally, since the centralized medical and biographical records system would not be the property of any one health care provider, the individual medical records could be owned by individual patients. Patients may authorize or deny access to their medical and biographical records or limit access to only portions of their medical record to specific health care professionals thereby controlling privacy of the patient and confidentiality of the patient's medical and biographical information. Patients also benefit by being able to add biographical information about themselves as well as review and comment on the contents of their records input by others for substance and accuracy.

A centralized electronic medical and biographical records and medical diagnostic system would also be beneficial in reducing health care costs and being a foundation upon which health care insurance programs may be based. By centralizing the medical history of a patient, reduced costs may be realized through avoiding repeating tests or prescribing medications or treatment that has been previously found to be unsuccessful or contraindicated. By reducing unnecessary treatment, health costs would be reduced, resulting in lower insurance premiums from insurers that would not have to cover unnecessary treatments.

U.S. Patent Application Publication No. 2014/0074509 teaches a dashboard user interface method which includes the steps of displaying a navigable list of at least one target disease, displaying a navigable list of patient identifiers associated with a target disease selected in the target disease list and displaying historic and current data associated with a patient in the patient list identified as being associated with the selected target disease including clinician notes at admission, receiving, storing, and displaying review's comments, and displaying automatically-generated intervention and treatment recommendations. One of the challenges facing hospitals today is identifying a patient's primary illness as early as possible, so that appropriate interventions can be deployed immediately. Some illnesses, such as Acute Myocardial Infarction (AMI) and pneumonia, require an immediate standard action or pathway within 24 hours of the diagnosis. Other illnesses are less acute but still require careful adherence to medium and long-term treatment plans over multiple care settings. The Joint Commission, the hospital accreditation agency approved by the Centers for Medicare and Medicaid Services (CMS), has developed Core Measures that have clearly articulated process measures. These measures are tied to standards that could result in CMS penalties for poor performance. To date, most reporting and monitoring of accountably measured activities are done after the patient has been discharged from the healthcare facility. The delay in identifying and learning about a particular intervention often makes it impossible to rectify any situation. It is also difficult for a hospital administrator to determine how well the hospital is meeting core measures daily. Clinicians need a real-time or near real-time view of patient progress and care throughout the hospital stay, including clinician notes that inform actions (pathways and monitoring) on the part of care management teams and physicians toward meeting these core measures. Case management teams have difficulty following patients' real-time disease status. The ability to do this with a clear picture of clinician's notes as they change in real-time as new information comes in during a patient's hospital stay would increase the teams' ability to apply focused interventions as early as possible and follow or change those pathways as needed throughout a patient's hospital stay, increasing quality and safety of care, decreasing unplanned readmissions and adverse events, and improving patient outcomes. The software has been developed to identify and risk stratify patients at highest risk for hospital readmissions and other adverse clinical events.

U.S. Pat. No. 6,954,730 teaches a method for assisting diagnosis and treatment of temporomandibular joint disease which includes the steps of recording physical symptoms, conducting a plurality of medical examinations related to temporomandibular joint disease, creating a diagnostic criterion based on conditions known to be a factor in diagnosis of temporomandibular joint disease and determining which of a plurality of patients match the diagnostic criteria.

U.S. Pat. No. 6,736,776 teaches a method which diagnoses and interprets dental conditions using a computer system. An image of the lesion being diagnosed is captured and terms describing the lesion are selected. A differential diagnosis list of the most probable lesions is returned. The user views details about each listed lesion until a match is selected, and appropriate medications for the selected lesion are presented. Medication details are reviewed and a proper medication to prescribe is selected. The user can generate a prescription, treatment algorithm, directions report, or a medication report. If the user is uncomfortable with the diagnosis, a referral report can be generated. For performing routine interpretation of dental conditions, the user captures an image for digital x-ray analysis. The user selects the task, such as caries detection, for which to optimize the image. The system optimizes the image based on the task selected and displays the optimized image.

U.S. Pat. No. 5,839,438 teaches a neural network system which diagnoses patients' medical-conditions, and which provides an efficient aid in identifying and interpreting factors which are significant in the medical diagnosis. The neural network is trained to recognize medical conditions by being provided with input data that is available for a number of patients, and diagnosis made by physicians in each case. Upon completion of a training period the neural network system uses input measurement and interview data to produce a score, or a graded classification, of a patient's medical condition that is accompanied with a diagnosis interpretation. The interpretation is a sorted catalogue of individual factors and interactions that influenced the score. The interpretive facility is based on comparison with a set of nominal values for each input factor or interaction. It can assist the physician in making a diagnosis of the patient's condition and can further provide a “second opinion” that may either confirm the physician's findings or point to ambiguities that call for a more detailed analysis.

U.S. Pat. No. 4,715,367 teaches a multifunctional behavioral modification device which diagnoses, treats, and monitors treatment for snoring, bruxism, or sleep apnea. Treatment consists of regulatable aversive shock, automatically occurring with each audible sound from snoring until snoring ceases or continuously but pulsatingly administered from clenching or grinding of teeth until the action ceases or continuously but pulsatingly administered from sleep apnea until breathing restarts. The placement of electrodes for administering the regulatable aversive shock is such to actuate a motor nerve thereby allowing use of a shock so mild as not to awaken a sleeper but sufficient to condition against the adverse behavior being sensed.

U.S. Pat. No. 8,417,010 teaches a method for diagnosis and evaluation of tooth decay which includes the steps of locating in an x-ray image the contour of the dento-enamel junction (DEJ), measuring optical density along contours substantially parallel to and on either side of the DEJ contour and calculating at least one numerical decay value from the measured optical densities. A method for diagnosis and evaluation of periodontal disease includes the steps measuring in an x-ray image a bone depth (BD) relative to the position of the cement-enamel junctions (CEJs) of adjacent teeth, measuring bone density along a contour between the adjacent teeth and calculating a numerical crestal density (CD) value from the measured bone density. Calibration standards may be employed for facilitating calculation of the numerical values. A dental digital x-ray imaging calibration method for at least partly correcting for variations of the optical densities of images acquired from the dental digital x-ray imaging system.

U.S. Pat. No. 5,742,700 teaches a caries detection method which quantifies a probability of lesions existing in tissues. Digital X-ray images are segmented and processed to generate feature statistics inputs for a neural network. The feature statistics include co-linearity measurements of candidate lesions in different tissue segments. The neural network is trained by back propagation with an extensive data set of radiographs and histologic examinations and processes the statistics to determine the probability of lesions existing in the tissues.

U.S. Pat. No. 7,324,661 teaches a computer-implemented system of intra-oral analysis for measuring plaque removal which includes hardware for real-time image acquisition and software to store the acquired images on a patient-by-patient basis. The system implements algorithms to segment teeth of interest from surrounding gum and uses a real-time image-based morphing procedure to automatically overlay a grid onto each segmented tooth. Pattern recognition methods are used to classify plaque from surrounding gum and enamel, while ignoring glare effects due to the reflection of camera light and ambient light from enamel regions. The system integrates these components into a single software suite with an easy-to-use graphical user interface (GUI) that allows users to do an end-to-end run of a patient record, including tooth segmentation of all teeth, grid morphing of each segmented tooth, and plaque classification of each tooth image.

U.S. Pat. No. 7,530,811 teaches a method which automatically separates tooth crowns and gingival tissue in a virtual three-dimensional model of teeth and associated anatomical structures and which orients the model with reference to a plane and automatically determines local maxima of the model and areas bounded by the local maxima. The method automatically determines saddle points between the local maxima in the model, the saddle points corresponding to boundaries between teeth. The method positions the saddle points along a dental arch form. For each tooth, the method automatically identifies a line or path along the surface of the model linking the saddle points to each other, the path marking a transition between teeth and gingival tissue and between adjacent teeth in the model. The areas bounded by the lines correspond to the tooth crowns; the remainder of the model constitutes the gingival tissue.

U.S. Patent Application Publication No. 2002/0143574 teaches a system which integrates mobile imaging units into an application service, and which provides for data storage and information system support. The system includes a mobile imaging unit including medical diagnostic equipment, a data center storing medical information in electronic form and a mobile imaging unit/data center communication interface allowing medical information transmission between the mobile imaging unit and the data center. The system also includes a healthcare facility and a healthcare facility/data center communication interface allowing medical information transmission between the data center and the healthcare facility.

U.S. Patent Application Publication No. 2010/0255445 teaches a system which plans and/or delivers an oral or facial endosseous implantation in a patient and which include a processing module, a surface imaging scan and a CT scan which utilizes a locator mouthpiece having a plurality of reference points thereon and can send scanned data to a treatment planning module. A processing module processes the data and the surface data into an output that includes three-dimensional (3-D) representation data indicative of at least one of an oral structure and a facial structure of the patient. A system includes a fabrication module that produces a physical model based on the 3-D representation data and indicating a planned location of an endosseous implant. A system includes a surgical module that guides implantation of an endosseous implant based on the 3-D representation data. The system may also provide a robotic implantation device which may assist the dental professional in placing the implant into the oral structure of an individual patient.

U.S. Patent Application Publication No. 2013/0144422 teaches a method which produces a dental implant surgical guide. A patient-specific virtual model is generated using image data specific to a patient and his virtual dental implants. The virtual model aligns the image data with the virtual dental implants using modeling software. A virtual mold is generated from the virtual model, and a physical mold is generated from the virtual mold. The physical mold is covered with a thermoplastic sheet via a thermoforming process. Excess thermoplastic material is trimmed off after the thermoforming process to produce a thermoformed piece. Metal tubes corresponding to each the virtual dental implants are placed onto the physical mold denoting the position, trajectory, and depth of the one or more virtual dental implants. A dental implant surgical guide that contains the thermoformed piece with the one or more tubes is produced.

US Patent Publication No. 2011/0287387 teaches a method for imaging the surface of a tooth which is executed at least in part on a computer records a first set of images of the tooth. Each image in the first set of images is illuminated according to a pattern oriented in a first direction. A second set of images of the tooth are recorded, wherein each image in the second set of images is illuminated according to a pattern oriented in a second direction that is shifted more than 10 degrees with respect to the first direction. A first contour image is reconstructed according to the recorded first set of images and a second contour image according to the recorded second set of images. A residual image is formed as a combination of the first and second contour images. The residual image is analyzed, and surface conditions of the tooth reported.

U.S. Pat. Nos. 8,478,698 and 8,856,053 teach a method which diagnoses and identifies a treatment for an orthodontic condition. The method generally entails the use of a server on which a centralized website is hosted. The server is configured to receive patient data through the website. The method includes the use of a database that includes or has access to information derived from textbooks and scientific literature and dynamic results derived from ongoing and completed patient treatments. The method also includes the operation of at least one computer program within the server, which can analyze the patient data and identifying at least one diagnosis of the orthodontic condition. The method entails assigning a probability value to the at least one diagnosis, with the probability value representing a likelihood that the diagnosis is accurate. The method further includes instructing the computer program to identify at least one treatment approach, a corrective appliance, or a combination thereof for the at least one diagnosis. Many methods have been developed or, more typically, envisioned which, hypothetically, could automate the capture of patient data and diagnosis of an orthodontic condition. These actual (or contemplated) methods employ certain components and subsystems that may automate the capture of patient data (such as orthodontic images or scans), the transfer of such data to an orthodontist, and/or even the interpretation of such data (or, more typically, discrete portions of such data). The currently available methods fail to include an ability to make decisions based on interpreted data, in an automated fashion. In other words, the currently available methods do not include an effective, accurate, and efficient “artificial intelligence” capability, in the automated diagnosis and treatment of an orthodontic condition. The server is configured to be capable of execute an artificial intelligence algorithm based on one or more inputs. The inputs are derived from patient data, information derived from textbooks and scientific literature and dynamic results derived from ongoing and completed patient treatments. The inputs include one utility value that indicates a relative importance of a treatment parameter versus other treatment parameters. The server instructs the computer program to identify a treatment regimen approach, a corrective appliance, or a combination thereof, for a diagnosis and is configured to estimate a treatment time for the treatment regimen. The artificial intelligence algorithm utilizes one of statistical estimation methodology, optimization methodology, control theory methodology and a combination thereof. A computer readable medium has instructions stored thereon that, when executed by a processor, causes the processor to perform a method which includes the steps of receiving patient data from a server on which a website is hosted, receiving information from a database that includes, or has access to, information derived from textbooks and scientific literature and dynamic results derived from ongoing and completed patient treatments and analyzing the patient data and identifying at least one diagnosis of the orthodontic condition based on the information derived from textbooks and scientific literature and the dynamic results derived from ongoing and completed patient treatments. The method also includes the steps of executing an artificial intelligence algorithm based on one or more inputs derived from at least one of the patient data, the information derived from textbooks and scientific literature and the dynamic results derived from ongoing and completed patient treatments and assigning a probability value to the at least one diagnosis. The probability value represents a likelihood that a diagnosis is accurate and identifies at least one treatment regimen for the at least one diagnosis. The treatment regimen includes one of a treatment approach, a corrective appliance, and a combination thereof. The probability value is assigned to the diagnosis in the computer readable medium and is based, at least in part, on a confidence level that has been assigned to a diagnostic data set which the server identifies as a statistical best fit for coordinates assigned to a tooth of the patient. The coordinates correlate to a location and position of the one tooth. The computer readable medium calculates a probability value that is correlated with a relative likelihood of the treatment regimen being effective to reorient at least one tooth of the patient. The inputs include one utility value that indicates a relative importance of a treatment parameter versus other treatment parameters.

Referring to FIG. 1 in conjunction with FIG. 2, U.S. Pat. Nos. 8,478,698, and 8,856,053 an automated diagnosis of an orthodontic condition begins with the production of patient-specific data which may include patient photographs 2, study models 4, radiographs 6 and/or combinations thereof. The types of data captured for a patient may either be the same for all patients or may be customized for each patient. The “orthodontic condition,” includes an arrangement of a patient's teeth that is undesirable according to applicable orthodontic standards. Such arrangement may be undesirable for medical, orthodontic, aesthetic, and other reasons. Such orthodontic conditions include, but are not limited to, overbites, crossbites, open bites, over jets and underbites. These patient data may then be provided to a server 8 through a centralized website 10.

Referring to FIG. 2 the patient data may be provided to the server 8 within the centralized website 10 through which the patient data may be uploaded and transferred to the server 8, or through a constant data feed through a standard Internet connection. The server 8 includes certain tools 12 for analysis and interpretation of the patient data and for making intelligent and probabilistic diagnosis and proposed treatments for an orthodontic condition. The server 8 can communicate with at least one database 14 (or group of databases). The database 14 stores and/or has access to knowledge and information derived from scientific, medical, and orthodontic textbooks and literature 16. A single database 14 either stores all of such information or, alternatively, stores portions of such information with the server 8 having access to additional information that is stored within other databases.

Again, referring to FIG. 1 in conjunction U.S. Pat. Nos. 8,478,698 and 8,856,053 the method employs a systematic approach to evaluating the strength of scientific evidence that may be retrieved from the database 14 described herein, for the purpose of diagnosing an orthodontic condition. The server 8 may consider the quality, quantity, and consistency of the evidence to derive a grade or confidence level of the available knowledge. Various criteria, such as indirect supporting evidence, may be considered in assessing the strength of each piece of scientific evidence. The scientific evidence may then be ranked, based on the grade levels (or confidence levels) assigned thereto. The method may consider the first highest grade or strongest evidence (i.e., evidence of higher-grade levels) being derived from at least one systematic review of one or more well-designed and randomized controlled trials. A second highest grade may be assigned to evidence derived from at least one properly designed randomized controlled trial, which involved an appropriate sample size and statistical power. A third highest grade may be assigned to evidence derived from well-designed trials, without randomization; a single group pre-post, cohort, time series study; or matched case-controlled studies. A fourth grade may be assigned to evidence from well-designed, non-experimental studies, carried out by more than one center or research group. A fifth and lowest grade of evidence may consist of opinions of respected authorities which are based on clinical evidence and/or descriptive studies or reports of expert committees. The database 14 further includes, or has access to, information that represents dynamic results from ongoing and previously completed orthodontic studies 18. These dynamic results 18 is organized by orthodontic condition, such that the most relevant information may be retrieved as quickly as possible, within the database 14. Similar to the information derived from scientific, medical, and orthodontic textbooks and literature 16. All the dynamic results 18 may be stored within the database 14 or, alternatively, portions thereof may be stored within the database 14 and other dynamic results 18 may be retrieved, as needed, from third party databases. Upon providing the server 8 with patient data including patient photographs 2, study models 4, radiographs 6, and/or combinations thereof, a user may instruct the server 8 to conduct an automated diagnosis. The automated diagnosis is based upon patient data, information derived from scientific textbooks and literature 16 and dynamic results from ongoing and previously completed orthodontic studies 18. The server 8 employs the use of logic-based rules and decision trees 20 to diagnose an orthodontic condition based on all of such information. The server 8 expresses the diagnosis by identifying one or more orthodontic conditions, along with a probability value for each orthodontic condition. The probability value represents the relative probability that the diagnosis is accurate. The server 8 is configured to output (recommend) one or more treatment approaches and/or corrective orthodontic appliances. For each diagnosis identified by the server 8, the server 8 proposes one or more treatment approaches, corrective appliances, or combinations thereof. Each proposed treatment approach and corrective appliance is correlated with a probability value. This probability value represents the probability of the proposed treatment approach and/or appliance correcting the diagnosed orthodontic condition. A user may input patient preferences and/or orthodontist-specified preferences to the server 8 through the centralized website 10. A patient may filter the proposed treatments and corrective appliance results based on cost, or the relative aesthetics of an appliance. An orthodontist may filter the proposed treatments and corrective appliance results based on his/her bias in that an orthodontist may instruct the server 8 to only consider not consider a certain type of corrective appliance. Upon completion of the foregoing process the server may be instructed to generate a report which summarizes the patient data, the diagnoses and associated probability values, the proposed treatment approaches and/or corrective devices (and the probability values associated therewith) and any patient and orthodontist preferences that were considered during the analysis. The server 8 is configured to analyze the patient data by identifying a location and position of a plurality of teeth in the patient data in either two-dimensional space or three-dimensional space provided that the type and amount of patient data provided to the server 8 is sufficient to do so. The server 8 may be configured to undertake this analysis automatically or the centralized website 10 provides users with certain on-line tools to specify the location and position of the plurality of teeth in the patient data. Such on-line tools may be used to identify, within the patient data, the location and position of a patient's incisors, canines, premolars and molars, as shown within the patient data that has been provided to the server 8. The location, position, contours and size of the plurality of teeth may be mapped out by such user within the centralized website 10. The user views the patient data that has been uploaded to the server 8 and uses a graphics tool that allows him to either approximately trace or identify the outer boundaries of each tooth. The server 8 may be further configured to assign coordinates to each tooth within the plurality of teeth. Such coordinates are correlated to the location and position of each tooth, as either automatically determined by the server or otherwise identified by a clinician, using the on-line patient data analysis tools. The coordinates for each of the plurality of teeth may then be compared by the server 8 to a table contained within the database 14. The table includes a series of diagnostic data sets, with each diagnostic data set including either coordinates or a range of coordinates which are correlated with a known location and position of a plurality of teeth and a previously diagnosed orthodontic condition which previous diagnoses are derived from information derived from textbooks and scientific literature and dynamic results derived from ongoing and completed patient treatments). The server 8 may then be instructed to identify a diagnostic data set contained within the database 14 that either represents a statistical “best fit” or most closely resembles the coordinates for the plurality of teeth of the patient. At this point the server 8 may be instructed to diagnosis the orthodontic condition based on the “best fit” diagnostic data set that it identified. The server 8 may further assign a probability value to this diagnosis. The probability value is based, at least in part, on a confidence level that has been assigned to the diagnostic data set which the server identifies as the statistical best fit for the coordinates for the plurality of teeth of the patient. This confidence level is influenced by the grade level that is assigned to the evidence that supports a connection between the orthodontic condition which is correlated with a particular diagnostic data set. The computer program housed in the server 8 may be instructed to identify at least one treatment approach, a corrective appliance, or a combination thereof for the at least one diagnosis that is derived from the patient's data. This step may be carried by instructing the server 8 to calculate a set of target coordinates which represent a desired and corrected location and position of each tooth in the plurality of teeth of the patient. Based on the target coordinates, the current location and position coordinates of the patient's teeth and the diagnosed orthodontic position the server 8 may be instructed to identify at least one treatment approach, a corrective appliance, or a combination thereof which will be effective to reorient the plurality of teeth towards the location and position represented by the target coordinates. The server 8 may further be instructed to calculate a probability value that is correlated with a relative likelihood of the at least one treatment approach, corrective appliance, or a combination thereof, being effective to reorient the plurality of teeth to a location and position represented by the target coordinates. The method employs certain additional algorithms in analyzing patient data, diagnosing orthodontic conditions and probability values therefor and proposing treatment approaches and corrective appliances and probability values therefor. The server 8 is configured to assign greater value/weight to existing scientific and medical knowledge, relative to dynamic results from ongoing and completed treatments when diagnosing and providing recommended treatment protocols for patients.

Artificial intelligence algorithms are employed in order to create an artificial neural network which enables the server to perform the orthodontic diagnosis, treatment planning and prognostication steps. The algorithms may utilize statistical estimation, optimization and control theory methodology, or combinations thereof. In the case of statistical estimation methods, estimators and estimation methods that may be employed include, but are not limited to, the following: maximum likelihood estimators, Bayes estimators, method of moments estimators, Cramer-Rao bound, minimum mean squared error (also known as Bayes least squared error), maximum a posteriori, minimum variance unbiased estimator, best linear unbiased estimator, unbiased estimators, particle filter, Markov chain Monte Carlo, Kalman filter, Ensemble Kalman filter and Wiener filter. The statistical optimization techniques that may be utilized include single-variable optimizations or multi-variable optimization techniques. The statistical optimization methods may include, but are not limited to, the following: Bundle methods, Conjugate gradient method, Ellipsoid method, Frank-Wolfe method, Gradient descent (also known as steepest descent or steepest ascent), Interior point methods, Line search, Nelder-Mead method, Newton's method, Quasi-Newton methods, Simplex method, and Sub-gradient method. The methods involve certain input provided by users so that the methods are dynamic. The algorithms employ control theory may be employed to solve problems in connection with the orthodontic diagnosis, treatment planning and prognostication steps. Non-limiting examples of such control theory methods include adaptive control, hierarchical control, intelligent control, optimal control, robust control and stochastic control. An important aspect of multiple optimizations is the handling of human preferences, such as the type of cost- and aesthetic-related preferences that a patient or orthodontist may provide to the system. Although selection or prioritizing alternatives from a set of available options with respect to multiple criteria termed Multi-Criteria Decision Making (MCDM) is an effective optimization approach, in practical applications, alternative ratings and criteria weights cannot always be precisely assessed due to unquantifiable, incomplete, and/or unobtainable information—or because of a lack of knowledge that may cause subjectiveness and vagueness in decision performance. As such, the application of fuzzy set theory to MCDM models provides an effective solution for dealing with subjectiveness and vagueness commonly found with clinical information. Human preferences—from both patient and clinician—may be assigned “utility values” in which a scaled real number is assigned to indicate its relative importance. The resulting weighting vector, which evaluates criteria of decision making, is then provided in fuzzy linguistic terms such as very poor, poor, fair, good, and very good. The method of decision tree algorithm for decision making in diagnosis and treatment planning is a decision tree method referred to as “C4.5,” and allows for input of continuous numerical data. Under this approach, a decision tree may be “learned” splitting a source set into subsets, based on an attribute value test. This process may be repeated on each derived subset in a recursive manner, which is completed when the subset (at a node) has the same value of the target variable, or when splitting no longer adds value to predictions. Decision trees are used for relatively simpler functions as decision-tree learners create over-complex trees (over-fitting), although pruning may, optionally, be performed to minimize this problem. In addition, concepts that are relatively more difficult to learn are not easily expressed by decision trees—and, in such case, more advanced algorithms are implemented in the methods described herein. Partially observable Markov decision processes (POMDPs) are used in clinical applications for decisions that are made based on incomplete information. POMDPs are advantageous insofar as they facilitate the combination of patient data derived from examination, photographs, radiographs, and any other diagnostic aids as well as the current state of knowledge of the cause-and-effect representation from these data and measurements. The feature selection may be performed using pattern recognition techniques. The treatment decisions with which to restore the patient to a more desirable or ideal state are produced.

H. Noroozi published an article entitled “Orthodontic treatment planning software,” in American Journal of Orthodontic Dentofacial Orthopaedics in June 2006 in Volume 129(6) on pages 834-7. New software can receive patient data in both graphic and numeric forms and then propose a treatment plan for nonsurgical orthodontic patients. The concepts of fuzzy logic enable the software to work with nominal parameters; the human brain is naturally accustomed to fuzzy variables. The computer program can propose treatment for some special cases, such as incomplete dentition.

A. El-Bialy presented a paper, entitled “Towards a Complete Computer Dental Treatment System,” at the Biomedical Engineering Conference on Dec. 18-20, 2008, in Cairo. The production of a 3D virtual clinic helps dentists in their treatment. To achieve this goal, different scientific areas are integrated such as computer graphics, pattern recognition, computer vision, information technology and finite element machine (FEM). The system includes a patient information system, automatic 2-D cephalometric, 3-D cephalometries, 3-D visualization, surgical planning, 3-D registration, soft tissue simulation, pre and post treatment analysis. Acquisition of the 3D virtual model of the patient is the foundation of this work. The CT slides of the patient's head are collected in a DICOM (Digital Imaging and Communication in Medicine) format. These slides are then compiled to build up the patient's 3D model. Using ray-casting volume rendering technique, a digital computer-based 3D replica is built. The theme also includes the detection of defective skeletal and dental areas by applying the appropriate diagnostic procedures. Based upon the diagnostic outcome, the necessary changes are executed; manipulation of the virtual 3D image and evaluation of the result after rectification is possible.

U.S. Pat. No. 7,991,485 teaches a computer-based method which constructs medical histories by direct interactions between the patient and which acquires pertinent and relevant medical information covering the complete life of a given patient. The method ensures that a complete lifelong medical history is acquired from every patient interacting with the health care system. Once acquired, the facts of the patient's life long and family medical history are analyzed automatically by databases to generate a set of the most reasonable diagnostic possibilities (the differential diagnosis) for each medical problem identified and for each risk factor for disease that is uncovered in the historical database. The automatically analyzed database of historical medical information is used as the search tool for bringing to bear on the diagnosis and treatment of each medical problem identified in each patient, the entirety of medical knowledge that relates to and can be useful for the correct and efficient diagnosis and treatment of each of every patient's medical problem. This collection of information is analyzed to generate a final diagnosis and treatment regimen.

U.S. Patent Publication No. 2002/0026105 teaches a patient analysis and risk reduction system which is used on a global network, and which includes a guideline database for storing a plurality of different medical guidelines for different health conditions, such as cardiovascular disease, and a patient information database. A risk evaluator evaluates patient information and generates a risk report based upon at least one of the different medical guidelines, and a risk reduction unit generates a physician's patient treatment plan based upon the different medical guidelines. Patient-specific instructions and educational material are also generated. A patient access unit permits patient monitored information to be entered by a patient while a clinician access unit permits patient reported information and clinician recorded information to be entered by a clinician via the global network. U.S. Pat. No. 7,698,154 teaches a system which provides a computerized medical and biographical records database and diagnostic information. A medical records database and diagnostic program is stored on a central computer that is accessible to individuals using remotely situated computers connected to a computer network. Individual patient medical and biographical records are owned by individual patients who can enter information in their record as well as grant or deny authorization to others, such as health care professionals, insurance providers and other entities, to review part or all of their record. The diagnostic program provides a series of diagnostic questions to an individual who must respond either “yes” or “no” to each question. Each potential response is weighted relative to its importance to a particular disease diagnosis. Relative weights for all responses to diagnostic questions are summed to identify potential diagnoses connected to the answered questions. The diagnostic program provides the individual with a list of potential diagnoses as well as permitting the individual to save the information to his or her individual medical and biographical record. The information maintained in the above system and process is utilized for health care financing and insurance. Medical record systems are well known in the prior art. Medical records have been used throughout the years of the practice of medicine in order to keep track of a patient's medical history, medical observations, diagnoses and any treatments prescribed to the patient. Often, a record contains information as to the success or failure of a particular treatment, a patient's allergies and reactions to drugs or treatments, and a record of patient visits. In addition to serving as a record of medical history and treatment, the medical record also serves as legal documentation of patient condition and treatment. Evolution of the health care system is engendering reevaluation of the roles of patients and health care providers with regard to access and content of medical records. Long term relationships and trust between a family doctor and patient are no longer commonplace because a change in residence, job or insurance carrier often requires the patient to change primary and/or specialty health care providers. Establishing relationships with a new health care provider can be tedious as medical records must first be transferred from previous health care providers and then reviewed by the new health care provider for history, therapies, and present therapeutic regimes. The new medical record being created by the new health care provider is often incomplete as patients frequently fail to remember to include all the necessary medical or biographical information. Patients sometimes convey erroneous information that can be ultimately detrimental to their health. Control of the information contained in a patient's medical and biographical record is also becoming a significant public issue and a source of controversy and stress. Health care professionals from different health care providers may not be able to easily review a patient's medical record and confer with each other as to diagnosis and treatment. This may be due to either security controls by the health care provider or by incompatible systems used by different health care professionals. Medical professionals wishing to confer with each other may be required to copy and mail or send a facsimile of the patient's record, introducing privacy and control issues. Current medical systems also often do not contain useful data such as family history, biographical data, genetic constitution or make-up, or other information that a patient may add to his or her medical record which could aid health care professionals in diagnosing the patient's condition or determine the best medical treatment.

U.S. Pat. No. 7,698,154 teaches a system which provides a computerized medical and biographical records database and diagnostic information. A medical records database and diagnostic program is stored on a central computer that is accessible to individuals using remotely situated computers connected to a computer network. Individual patient medical and biographical records are owned by individual patients who can enter information in their record as well as grant or deny authorization to others, such as health care professionals, insurance providers and other entities, to review part or all of their record. The diagnostic program provides a series of diagnostic questions to an individual who must respond either “yes” or “no” to each question. Each potential response is weighted relative to its importance to a particular disease diagnosis. Relative weights for all responses to diagnostic questions are summed to identify potential diagnoses to connected to the answered questions. The diagnostic program provides the individual with a list of potential diagnoses as well as permitting the individual to save the information to his or her individual medical and biographical record. The information maintained in the above system and process is utilized for health care financing and insurance.

Medical record systems are well known in the prior art. Medical records have been used throughout the years of the practice of medicine to keep track of a patient's medical history, medical observations, diagnoses, and any treatments prescribed to the patient. Often, a record contains information as to the success or failure of a particular treatment, a patient's allergies and reactions to drugs or treatments, and a record of patient visits. In addition to serving as a record of medical history and treatment, the medical record also serves as legal documentation of patient condition and treatment. Evolution of the health care system is engendering reevaluation of the roles of patients and health care providers about access and content of medical records. Long term relationships and trust between a family doctor and patient are no longer commonplace because a change in residence, job, or insurance carrier often requires the patient to change primary and/or specialty health care providers. Establishing relationships with a new health care provider can be tedious as medical records must first be transferred from previous health care providers and then reviewed by the new health care provider for history, therapies, and present therapeutic regimes. Also, the new medical record being created by the new health care provider is often incomplete as patients frequently fail to remember to include all the necessary medical or biographical information. In fact, patients sometimes convey erroneous information that can be ultimately detrimental to their health. Control of the information contained in a patient's medical and biographical record is also becoming a significant public issue and a source of controversy and stress. Presently, such records are treated as being “owned” by the medical offices or institutions in which the records are housed. Distrust on maintenance of confidentiality results in failure to disclose information that may be important for health-care decisions. This distrust may be increased as patients transfer to new health care providers. Medical record systems usually consist of handwritten notes, pictures, and documents created by a medical and health care professional. Recently, computer programs and systems have become available for the generation, storage, and retrieval of medical records. In general, such systems operate on a computer owned by a hospital or other health care provider and may only be accessed by health care professionals that are affiliated with the health care provider. Patient medical information is typically input into a medical record by a physician, nurse, or other health care professional. Several automated medical record systems have been designed and marketed in the health care field. U.S. Pat. No. 5,277,188 discloses a clinical information reporting system having an electronic database including electrocardiograph related patient data. Similarly, U.S. Pat. No. 5,099,424 discloses a computer system for recording electrocardiograph and/or chest x-ray test results for a database of patients. U.S. Pat. No. 4,315,309 discloses a patient report generating system for receiving, storing, and reporting medical test data for a patient population. U.S. Pat. No. 3,872,448 likewise discloses a system for automatically handling and processing hospital data, such as patient information and pathological test information using a central processing apparatus. In U.S. Pat. No. 5,065,315, a computerized scheduling and reporting system is disclosed for managing information pertinent to a patient's stay in the hospital. Also, U.S. Pat. No. 5,924,074 discloses an electronic data processing system. While present automated systems may provide electronic storage of medical data, they typically suffer from significant shortcomings that have plagued medical record systems since their inception. These systems, like their paper record counterparts, are typically only available to health care professionals affiliated with the hospital, clinic, or other health care provider that owns the medical record software program and computer system. The information contained in a patient's medical record would not be able to be reviewed by another health care professional who is not affiliated with the health care provider that maintains the medical record software. This becomes an issue for patients who choose to be treated by a different health care provider or who may require treatment while traveling in a location not served by their usual health care provider. Treatment may be prescribed which has been previously determined to be ineffective or which is contraindicated for the patient. Similarly, health care professionals from different health care providers may not be able to easily review a patient's medical record and confer with each other as to diagnosis and treatment. This may be due to either security controls by the health care provider or by incompatible systems used by different health care professionals. Medical professionals wishing to confer with each other may be required to copy and mail or send a facsimile of the patient's record, introducing privacy and control issues. Since the existing systems are “owned” by the health care provider, a patient may be kept from reviewing his or her own medical record for the substance or accuracy of its information. A patient cannot prevent, or control private information contained within the patient's medical record from being seen by any individual that has access to medical records, regardless of whether the individual has any right or need to review a particular portion of the patient's medical record. As such, information which the patient wishes to remain private may be reviewed, thereby compromising the patient's privacy, and potentially introducing a negative bias to the health care professional towards the patient. An example of such information may include past treatment for a sexually transmitted disease or sexual dysfunction that may be irrelevant to a particular medical specialty. Current medical systems also often do not contain useful data such as family history, biographical data, genetic constitution or make-up, or other information that a patient may add to his or her medical record which could aid health care professionals in diagnosing the patient's condition or determine the best medical treatment.

Presently available medical records systems are not suited for providing medical diagnoses. Advancements in automation, research, specialization, and medical knowledge have permitted modern day health care to be increasingly improved over the care provided in the recent past. While these advancements have resulted in improved success rates of medical treatment, individuals often delay seeking medical attention due to fear of the unknown and the inconvenience of being referred to multiple physicians. Patient referrals typically occur when a primary care physician makes a general diagnosis, then refers a patient to a physician specializing in the diagnosis. Further referrals may occur if the patient is referred to medical sub-specialties for further diagnosis and treatment resulting in additional patient cost, time, and inconvenience. Patients who face these inconveniences and costs or who have experienced them in the past may delay seeking treatment in the hope that a condition may simply go away thereby precluding the need to seek the help of a health care professional. This delay can cause a medical condition which could be easily treated early in its development to require longer treatment, or the condition may even become untreatable by the time medical assistance is sought. If the same patients were informed of potential diagnoses of their conditions, they can be aware of the risks of delaying medical assistance and may be persuaded to seek help earlier. Informed patients may even be able to reduce the inconveniences of multiple referrals by initially seeking the assistance of a health care professional who specializes in treating their condition.

Medical information is readily attainable to the public through medical books available in libraries and bookstores, medical phone help or “Ask-A-Nurse” telephone services, audio visual informational programs on television and videotape, and Internet sites specializing in medical care. The amount of available information can be overwhelming to an individual trying to determine the identification of his or her health condition who is unfamiliar with researching health information or who lacks a scientific background. Computer programs have been developed to provide individuals potential diagnoses based on their responses to a series of health-related questions. U.S. Pat. Nos. 5,910,107 and 5,935,060 describe diagnostic programs which can be accessed over a telephone or computer network. An individual is asked a series of weighted questions concerning the individual's health symptoms and can respond with “yes,” “no,” or “not sure” answers or may be asked to answer multiple-choice questions. From the responses, the program identifies a list of potential diseases which are indicated by the individual's health symptoms. U.S. Pat. No. 5,572,421 discloses an electronic medical history questionnaire in which a patient can respond “yes,” “no,” or “not sure” to medical questions. The questionnaire then provides the physician with suggested tests that may be performed and conclusions regarding the patient's health. U.S. Pat. No. 5,839,438 discloses a diagnostic system using a neural network to provide a patient diagnosis to a physician from input data comprising measured and interview data regarding the patient's condition. The diagnosis is based upon a databases of physician diagnoses of medical conditions and their corresponding symptoms. While prior art automated medical diagnostic programs diagnose a condition or confirm a diagnosis made by the physician, they are usually designed to be used by a physician and not a patient. The language and phrasing in these programs are designed for a medical professional and contain esoteric medical and health terms. Most patients do not understand these terms and therefore cannot effectively use the programs. The diagnostic information provided by these programs does not inform individuals of their various conditions before they seek medical assistance. A further shortcoming of prior art automated diagnostic programs is that they can accept input data that is often erroneous or not helpful. As an individual may select “not sure” or other answers which are not simply “yes” or “no,” an individual is able to avoid answering conditions they feel are minor are irrelevant, but which may provide helpful data if the individual were forced to select only a “yes” or “no” response. A software program designed to accept objective data and provide individuals with diagnostic information about their health conditions would be desirable. It would be beneficial to patients and health care professionals alike to develop an individual patient self-generated, fully controlled, and censored, centralized electronic medical and biographical records and medical diagnostic system that may be accessed by patients and health care professionals regardless of their affiliation with a particular hospital, clinic, or other health care provider. The medical and biographical records and medical diagnostic system would be maintained, stored, and delivered by a totally independent institution, not necessarily affiliated with the government, insurance, or health care industry. By using common language and phrasing tailored to different levels of education and familiarity with medical and health terms an individual could effectively utilize such a system to determine potential diagnoses prior to seeking medical attention, permit the individual to be better informed as to the potential medical specialty from which to seek assistance, and control the content of and access to the individual's medical record. A self-generated record of present illness and pertinent information would also benefit individuals by allowing them ample opportunity to ponder and respond without encumbrances from health care providers presence. Such presence often generates discomfort or uneasiness and may lead to confused, unconsciously withheld, consciously suppressed information (e.g., suppressed for fear of embarrassment) or miscommunicated medical and biographical information. A centralized electronic medical and biographical records and medical diagnostic system would also permit any health care professional to be aware of all of a patient's biographical and medical history that is relevant to treating the patient. Since the centralized medical and biographical records system would not be the property of any one health care provider, the individual medical records could be owned by individual patients. Patients may authorize or deny access to their medical and biographical records or limit access to only portions of their medical record to specific health care professionals thereby controlling privacy of the patient and confidentiality of the patient's medical and biographical information. Patients also benefit by being able to add biographical information about themselves as well as review and comment on the contents of their records input by others for substance and accuracy. A centralized electronic medical and biographical records and medical diagnostic system would also be beneficial in reducing health care costs and being a foundation upon which health care insurance programs may be based. By centralizing the medical history of a patient, reduced costs may be realized through avoiding repeating tests or prescribing medications or treatment that has been previously found to be unsuccessful or contraindicated. Therefore, by reducing unnecessary treatment, health costs would be reduced, resulting in lower insurance premiums from insurers that would not have to cover unnecessary treatments.

Referring to FIG. 3 a voluntary automated medical and biographical and diagnostic provides medical diagnostic information in which the patient obtains a list of potential medical diagnoses corresponding to input health symptoms. An individual patient's medical and biographical record information can be accessed, added, modified, maintained, and controlled by the patient. The voluntary automated medical and biographical and diagnostic system 100 includes a central computer 102 that is connected to a global computer network 104 (e.g., the Internet). The central computer 102 also has access to a medical and biographical records database 106 that contains a plurality of medical and biographical records 112 for individual patients. Also connected to global computer network 104 are a plurality of patient computers 108 and health care computers 110. Patients obtain access to their medical and biographical records by accessing central computer 102 via patient computers 108 connected to global computer network 104. The central computer 102 executes security program 114 that limits access to medical and biographical database 106 and individual medical and biographical records 112 contained therein. Once a patient's identity is verified by security program 114, the patient may gain access to his or her own individual medical and biographical record 112. Similarly, health care providers obtain access to patients medical and biographical records by accessing central computer 102 via health care computers 110 connected to global computer network 104. Central computer 102 executes security program 114 to limit access to medical and biographical database 106 and individual medical and biographical records 112 contained therein to health care providers that are authorized by a patient to access the patient's medical and biographical record 112. Individuals, whether patients, health care providers, or simply individuals interested in inquiring about a health condition, may execute medical diagnostic program 116 by accessing central computer 102 via either patient computers 108 or health care computers 110 connected to global computer network 104. Results from the execution of medical diagnostic program 116 are provided by central computer 102 to either patient computers 108 or health care computers 110 via global computer network 104. The creation and maintenance of medical records, including recording and correlating past medical history and biographical information; integrating genetic, laboratory, radiologic, and imaging results, prescribed medications, and treatments; noting patient allergies, reactions, and treatment outcome; updating medical records; emergency recalling medical records; and making medical records available and transportable is a very detailed and involved task. Extreme care is required to preserve and protect all the information contained in medical records as well as ensure that authorized personnel can retrieve information when it is requested. The task is complicated because of the difficulty in obtaining, maintaining, and correlating the information as well as providing security measures to protect access to the information. Solving these multiple difficulties while organizing the system to provide a user-friendly program provides substantial benefits to patients and health care professionals. With the extensive and rapidly increasing pharmaceutical armamentarium available today, it has become difficult for health care providers and patients to be aware of all the drugs taken in the past and the present, as well as their generic equivalents, interactions, and side effects. This is further compounded by the increasing inclusion of over-the-counter drugs, herbal treatments, and the like that many patients' intake regularly but do not consider as part of their “medicines” and therefore neglect to inform their health care providers that they are taking such substances. By providing a central registry and a rapid individualized analysis and correlation system, prompt warnings regarding interactions, side effects, and previous use (including effectiveness or lack of efficacy), can be extremely useful and beneficial. Another important contribution of a central registry is the ability to differentiate between intolerance, side effects, or true allergies of patients to drugs. With the ever-increasing mobility of patients and families, the breakdown of roots and family connections, and advances in science, ready access to extensive knowledge of a patient's genealogy, genetics, environmental and biologic events become important and sometimes crucial in the differential diagnoses and therapy. Knowing the genealogy and place of origin of a patient may facilitate locating someone with similar genetic makeup for organ or tissue acquisition or transplantation (i.e., stem cells, etc.). An example would be an environmental exposure, discovered many years after the event and its correlation to diseases or conditions, that appear unrelated until the correlation is made of biography, location, and exposure. For the patient, the benefits include (1) ready availability of a chronological register of a patient's lifelong medical history, (2) full control of access to personal information, (3) the ability to restrict personal facts or areas of information, (4) ready availability of an electronic, free, easily accessed, confidential, personal medical consultant for health condition diagnosis, (5) a potential reduction in the need for medical services thereby saving money and inconvenience, (6) protection from conflicting therapies, (7) unbiased health care and insurance referral service, and (8) portability of medical history and biographical information between health care providers. The voluntary automated medical and biographical and diagnostic system 100 provides medical and biographical records database 106 which contains medical and biographical records 112. Unlike the medical records of the prior art which are the property of individual health care providers such as doctors, clinics, hospitals, and the like, the medical and biographical records 112, or folios, of the voluntary automated medical and biographical and diagnostic system 100 are the property of the patients who are the subject of the folios. The patients, being the owner of their own folios, can review what is in their folios. They also control access to their folios in part or in total. A female patient may provide a family doctor with authorization to access general health information in her folio but prevent her family doctor from accessing information she permits only her gynecologist to review. The voluntary automated medical and biographical and diagnostic system 100 contains medical and biographical records 112 that are the property of individual patients, it may also contain medical records and biographical records 112 that are owned by individual health care providers such as doctors, clinics, hospitals, and the like. A health care provider may to archive some or all of its own records on system 100 and benefit from its central access, security provisions, and other features. In contrast to a folio of an individual patient, the health care provider's archive may contain folios of numerous individual patients that receive treatment from the heath care provider. Patients and health care providers may add both medical and biographical patient information such as physical examinations, genetic constitution and history, social history, mental and emotional health history, organ system history, surgical history, environmental history, dental and oral health history, laboratory results, radiological and imaging history, treatment therapies and medications history, ontological and ophthalmological history, past history of prior injuries, patient health related events, job related health issues, chemical exposures, temperature, metabolic profiles, organ function tests, biochemical, anatomical, physiological, and pathological histories, alternative medicines, and so forth. Entered information may also include family history, or any other information the patient desires to be contained in his or her folio. The patients may review entries made by their health care professionals. While the patients cannot delete authorized entries by health care professionals, they may add comments that they feel are necessary to clarify an entry. The folios are stored as records 112 in database 106 associated with central computer 102. Central computer 102 is connected to a computer network, preferably global computer network 104. Patients and the people they authorize can access a patient's folio via computers 108 positioned remotely from central computer 102 that are also connected to computer network 104. A significant benefit of the voluntary automated medical and biographical and diagnostic system 100 is the ability for patients, and the people that they authorize, to access medical and biographical information stored in a patient's folio regardless of the affiliation of the patient or the medical professional to a particular clinic, hospital, or other health care provider. This is significant as a patient's folio may be accessed regardless of whether he or she is being treated at a local health care provider or a remote health care provider such as when a patient is injured while traveling or on vacation. The voluntary automated medical and biographical and diagnostic system 100 can also be modified to provide information in different languages and to translate information from one language to another. English language text and information can be translated to Spanish to permit Spanish-speaking individuals to effectively use the system. Another benefit of the voluntary automated medical and biographical and diagnostic system 100 is the ability to maintain a complete medical and biographical record over a patient's life. Medical records of the prior art are typically in varying states of completeness and reside at various health care providers that a patient has used over his or her lifetime. In contrast, the voluntary automated medical and biographical and diagnostic system 100 provides a centralized medical and biographical record database, which could be used by all health care providers when treating the patient. As is well known, even the most interested and compulsive of people lack the discipline and perseverance to maintain a record of their lives. Many important pieces of data are forgotten, remembered inaccurately, or confused chronologically. Allowing long-term compilation of data facilitates chronological or correlative analysis which presently is mostly non-existent. The patient's folio would be essentially complete and selectively accessible to any authorized physician, dentist, or other person. Each record 112 includes one or more sectors of related medical and biographical information. Although each sector preferably includes individual and independent units, the voluntary automated medical and biographical and diagnostic system 100 provides correlative activity between parts of the individual and independent units in a controlled manner. Access to information in each unit is limited to authorized individuals while at the same time serving the needs of all the potential users of the voluntary automated medical and biographical and diagnostic system 100. In addition to information contained in patient medical and biographical records 112, system 100 preferably provides information that is useful to both the patient and physician. Hyperlinks to scientific and medical sources, medical information relating to health signs and symptoms, diagnostic references, medical and surgical therapies, and medical, pharmaceutical, and scientific dictionaries and thesauruses, are available in this embodiment for as much inquiry as desired. Hyperlinks can be designed to provide a progressive hierarchy to satisfy different levels of sophistication. Those skilled in the art understand many techniques for providing linking and searching via global computer network 104. The voluntary automated medical and biographical and diagnostic system 100 may include a variety of systems and processes to achieve an automated medical record, diagnosis, and treatment system and method that is patient owned and controlled. The following are examples of some systems and processes which may be included in the voluntary automated medical and biographical and diagnostic system 100: registration; identification; a security process and system to allow or deny accessibility to the medical and biographical records; entry of medical history; recording information in medical and biographical records (e.g., medical history; physical examination; anatomical, biochemical, physiological, pathological, and laboratory tests; and radiology and imaging information among other information); analysis and correlation of health symptoms; accessing disease and symptom oriented treatises such as the Merck Medical Manual, medical journals, and so forth; diagnosing medical conditions based upon weighting patient responses to diagnostic questions according to the relevancy of the answers to a particular disease or condition; providing an individual with therapeutic recommendations; recording actual medical therapies prescribed to a patient; predicting patient outcome to a given therapy; recording actual outcome to a given therapy; mental and emotional health and counseling; electronic dermatological evaluation which may include a dermatopathology atlas; electronic ophthalmologic evaluation and atlas; dental and oral care and surgery; providing an individual with social and welfare services; cumulative recording of radiological and imaging studies; cumulative recording and correlation of anatomical, biochemical, physiologic, pathologic, and laboratory studies; referral to health care provider; evaluating a health care provider; monitoring health care provider; notifying a patient of medical due dates; developing a genealogy tree; developing a patient's genetic constitution and history; providing access to medical, pharmaceutical, biologic, scientific dictionaries, thesauruses, etc.; acquisition and evaluation of audio and/or video information from directed self-examination; acquisition and evaluation of biologic parameters and electronic information and examinations; insurance program registration and automatic updating; and primary and specialist information interchange. The preferred process and system requirements include immense data collection and correlation capability, easy portal accessibility, a multilevel security system, data entry and periodic upgrading by multiple health care providers, and acquisition of proprietary information and sources. Referring now to the registration process, each patient will own his or her personal unique folio. The voluntary automated medical and biographical and diagnostic system 100 permits only the patient, or his or her representative (e.g., parents of an underage child), to register. It is envisioned that eventually most patients will be registered at birth and the folio containing the patient's life medical history being maintained henceforth. To maintain a unique folio for each patient and to ensure that only the patient and those to whom the patient has granted authority will have access to the patient's folio. This may be done by requiring an identification sequence to be input wherein identifying data unique to the patient is required to access a folio. Examples of such identifying data include 1) full name of the patient, without abbreviations, 2) state or country of birth, 3) birth date (dd/mm/yyyy), 4) patient social security number (SSN), and 5) a personal identification number (PIN). As advances in technology permit, the identity of a patient may be verified by physical identifiers. Examples of such identifiers, also referred to as biometrics identifiers, include 1) fingerprint(s), 2) retinal or ocular image, 3) voice pattern (with or without a key verbal code), 4) DNA or generic print, and 5) biochemical or blood type (AB, Rh, etc.). In addition, depending on the level of security desired, an electronic signature may be required of the patient or registrant to enhance security of the identification sequence. The signature can be requested at the time of registration or at the end of the interaction to add further recognition of the validity of the included information, or as a legal validation of the preceding text. With the previously described registration and identification steps, the affixed signature at the end of the document, including the option of requiring a repetition of the identification sequence, would improve the security of data as well as provide an electronic signature for legal purposes. The identifying data references are keyed to a unique number that has been randomly assigned to the patient upon initial registration. This number is randomly assigned to prevent a folio from being correlated to a particular registration date, patient name, or other information that may indirectly identify the identity of a particular patient. The unique number in turn references the patient's folio. The folio does not contain the actual patient identification data, but rather just the unique number. The separation of patient data from medical and biographical data and the requirement of a randomly assigned number increases the security of a patient's medical and biographical information from being accessed by an unauthorized individual breaking into computer 102 and its associated database 106 used to store the patient's folio. If unauthorized access is gained by someone breaking into the voluntary automated medical and biographical and diagnostic system 100, all that could be accessed would be medical and biographical data that is anonymous except for the unique randomly assigned number. By maintaining separation of a patient's identifying data from his or her actual medical and biographical data, medical and biographical data can be studied for information with full preservation of patient anonymity. This way longitudinal and population studies can be performed without compromising the confidentiality of a patient's medical and biographical record. The PIN or biometric key permits the patient or an individual authorized by the patient to obtain access to the folio containing the patient's recorded medical and biographical data. Additional security procedures of the voluntary automated medical and biographical and diagnostic system 100 may be implemented such as requiring reentry of the patient's PIN or biometric data for opening the folio or secured portions within the folio. Copying of records without proper authority (i.e., without the patient's PIN or biometric verification) would be attached to a “cookie” that would eliminate or scramble the unauthorized copied data from any files where the data was copied. A “cookie” is a small program or file that executes a specific command, such as delete or scramble a file, by utilizing the computer into which the program has been imported for command execution.

Health care providers may also wish to take advantage of the security offered by the registration and identification sequence requirements provided by the voluntary automated medical and biographical and diagnostic system 100 to store their patients' records. A health care provider may store multiple records of its patients. In this scenario, the identification sequence is the same as for any registrant, whether a health care provider or a patient. Once entered the individual health care provider archive, access is granted to each one of the files the health care provider registrant has generated or stored in medical and biographical records database 106. The individual files contain only the information that the health care provider has specifically included in these files but requires the active participation or an affirmative action of the health care provider for the inclusion of information to occur. This affirmative action confirms the positive desire of information inclusion, therefore negating the possible health care provider assertion of ignorance of information inclusion. Access to each one of the patient records in the medical and biographical records database requires re-g identification of the health care provider (by whichever measure is established by the health care provider) before opening the individual file. This additional step circumvents the possibility of unauthorized access to the files of the medical and biographical records database if it is inadvertently left open by the health care provider. The voluntary automated medical and biographical and diagnostic system 100 protects the health care provider's records or files from being forwarded to another file or database by requesting specific authorization for forwarding by the individual registered as the subject of the file. This extra measure complies with the requirements of the Health Insurance Protection and Portability Act (HIPPA). The specific authorization restriction of HIPPA can be avoided by transferring the responsibility for the information to the individual, for the individual's personal files. The assumption is that all the health care information (excluding financial and other types of documents) contained in the health care provider files should be also included in the individual's personal file.

U.S. Patent Application Publication No. 2013/0122468 teaches a method which analyzes and displays an oral health status of a tooth, or an entire dentition based on a measurement with a diagnostic device. Diagnostic data pertaining to a selected tooth, tooth surface, section of tooth surface or numbers of teeth in a mouth is recorded from an oral health diagnostic device, optionally along with an image of the tooth or tooth surface examined. The diagnostic data is processed and compared with reference data to determine an oral health status of the tooth. The oral health status of the tooth is then displayed on an odontogram shown in a user interface. The user interface may also provide reports comparing changes in the measured data and/or images along with the therapies used, thereby enabling the measurement, and tracking of outcomes from various therapies over time. With the widespread use of fluoride, the prevalence of dental caries has been considerably reduced. Nonetheless, the development of a non-invasive, non-contact technique that can detect and monitor early demineralization and or carious lesions on or beneath the enamel, dentin, root surface or dental restorations, is essential for the clinical management of this problem. A number of different diagnostic devices and methods have been developed to meet this need, including laser-induced fluorescence of enamel or to the fluorescence caused by porphyrins present in carious tissue [R. Hibst, K. Konig, “Device for Detecting Dental Caries”, U.S. Pat. No. 5,306,144 (1994)] and photothermal radiometry [A. Mandelis, L. Nicolaides, C. Feng, and S. H. Abrams, “Novel Dental Depth Profilometric Imaging Using Simultaneous Frequency-Domain Infrared Photothermal Radiometry and Laser Luminescence”, Biomedical Optoacoustics. Proc SPIE, A. Oraevsky (ed), 3916, 130-137 (2000), L. Nicolaides, A. Mandelis, and S. H. Abrams, “Novel Dental Dynamic Depth Profilometric Imaging Using Simultaneous Frequency-Domain Infrared Photothermal Radiometry and Laser Luminescence”, J Biomed Opt, 5, 31-39 (2000), and R. J. Jeon C. Han A. Mandelis V. Sanchez S. H. Abrams “Diagnosis of Pit and Fissure Caries using Frequency Domain Infrared Photothermal Radiometry and Modulated Laser Luminescence” Caries Research 38, 497-513 (2004)[smooth surface and interproximal lesion detection].

While these oral health diagnostic devices succeed in providing quantitative measures of existing and anticipated oral health decay, their results are often not directly amenable to clinical practice. Firstly, the recording of numerical data based from a diagnostic device presents a workflow challenge to an oral health provider, and the manual recording of results is susceptible to transcription errors that could result in costly or inappropriate treatment. Secondly, describing and transcribing the status of oral tissues including exact color, shape and position of a pathological condition is most challenging and may lead to inaccuracies and inability to track changes in the tissues over time. Furthermore, merely sharing a numerical value provided by a diagnostic device with a patient offers little insight to the patient in terms of the severity of a problem. Such raw and direct results do not assist in providing a path that the patient and provider can take together to manage a given condition and/or mitigate risks of developing an oral health problem in the future.

U.S. Pat. No. 7,474,932 teaches a system which is for interactive three-dimensional dental imaging, and which provides for interactive computer-aided design (CAD) in dental applications. An interactive dental computer-aided design (CAD) system includes a graphical user interface for displaying at least one three-dimensional (3D) image for viewing by an operator, an access interface for receiving input from the operator, and a prosthesis design module providing design tools for creating a virtual 3D model of a dental prosthesis responsive to operator input. While a significant number of people have dental conditions that require replacement prostheses (e.g., crowns), many of these people elect not to have dental prostheses work performed because such work is traditionally costly, time consuming, and sometimes ineffective. Conventional dental practice methods require that a person make at least two separate visits to a dentist for replacement prostheses work—typically a first visit for diagnosis, planning and preparation work, and a second visit for installation and fitting. The person may also be required to wear a temporary prosthesis between visits. At the first visit, diagnostic work is performed to determine, with the patient's approval, a choice and method for treatment. In the context of a replacement prosthesis being a crown, the diagnostic work often includes taking diagnostic impressions (e.g., a wax mold) of the patient's teeth for a diagnostic study of the patient's dentition. Next, the patient's tooth structure is modified in preparation to “fit” a crown. The tooth that is to receive the crown is reduced in size such that the crown will “fit” on the tooth and within the patient's dentition. A physical dental impression of the prepared tooth is taken, and a temporary crown is placed over the tooth. The dental impression is sent to a dental laboratory (usually offsite), where technicians manually design a final crown based on the dentist's prescription and the patient's physical dental impression. Typically, the final crown design is performed manually on cast stones, which is a physically labor-intensive practice that can introduce errors into the final crown. The final crown is then manufactured and sent to the dentist. With the final crown ready, the patient is recalled for the second visit, which may be scheduled days or even weeks after the first visit. At the second visit, the temporary crown is removed, and the final crown is fitted, adjusted, and cemented into place. If for some reason the final crown does not fit properly, the patient may be required to repeat the preparation process described above and return at a later date for yet another visit. It has been found that a significant number of crowns manufactured using the above-described traditional techniques do not fit properly at the first installation, and thereby lead to repeat visits. As can be seen, traditional restoration treatment processes are long and time-consuming for both the patient and the dentist. Traditional dental restoration procedures also suffer from additional shortcomings. Conventional procedures for taking physical dental impressions and designing restoration prostheses from the physical impressions are prone to distortions that can affect the final fit of restoration prostheses. These distortions can be caused by numerous factors, including technique, temperature, manual handling, technician or dentist error, patient movement, material properties and age, or salivary contamination. Conventional procedures for designing restoration prostheses from physical dental impressions are labor intensive, time consuming, and costly. Prostheses are usually designed and fabricated offsite, which requires transport arrangements, costs, and time. As mentioned above, a patient may be required to wait significant amounts of time before returning to the dentist to have restoration prostheses fitted and installed. In sum, traditional dental restoration procedures are inefficient, error-prone, time consuming, labor intensive, and costly.

U.S. Pat. No. 10,624,601 teaches a cloud-based imaging protocol manager which pushes standard imaging protocols from the cloud to imaging devices registered with the protocol manager. The protocol manager maintains a library storing standard imaging protocols, determines whether an imaging device is compatible with the standard protocol(s) to be pushed, creates a push command which requests pushing the standard protocol(s) to a compatible imaging device, stores the push command in a command queue, converts the standard protocol(s) to raw protocol(s) usable by the imaging device. The imaging device polls the command queue to receive the push command, downloads the raw protocol(s) from the protocol manager, commits or refuses to commit the downloaded protocol(s), and sends a notification to the protocol manager indicating execution status of the push command. Imaging devices (e.g., magnetic resonance (MR) scanner, computed tomography (CT) scanner, X-ray acquisition system, positron emission tomography (PET) scanner, nuclear medicine (NM) scanner, etc.) use imaging procedures to obtain image data of a target, such as a patient. An imaging procedure is associated with one or more imaging protocols that define image acquiring and/or processing actions or elements, such as one or more imaging parameters, one or more scanning planes in which image(s) are to be captured, and so on. An imaging protocol may include parameters for an imaging device, such as tube current, tube voltage, filter usage, filter type, scan speed, etc. An imaging protocol may define a scanning plane for the associated imaging procedure, specify position and orientation of anatomical structure(s) or region(s) of interest in the patient, etc. An imaging protocol may further specify limits and/or other guidance on image noise, spatial resolution, and image texture including edge sharpness, artifacts, and radiation dose. An imaging device maintains a protocol database which stores various imaging procedures and/or protocols for the device to use according to one or more scenarios, reasons for examination, etc. The scenarios for examination may include patient size, anatomy type (e.g., heart, lung, kidney, brain, etc.), position, task, etc. Imaging protocols can be constructed for clinical tasks. A task function such as tumor detection, tumor sizing, vessel sizing, etc., can be incorporated into an objective function to determine a dose distribution for a given task and to find a minimum possible dose for a given performance level. During protocol development, results from similar clinical tasks (e.g., tuning for a given anatomical location, etc.) can be used to inform initial parameter selection for another clinical task (e.g., bone imaging in the wrist may be used to inform the initial selection of parameters for bone imaging in the ankle, etc.). Protocols for similar scenarios and tasks may vary on different brands/models of imaging devices. As an example, a protocol for a liver scan by imaging scanner A indicates a 120 kV tube current at 300 mA for 1 second. Scanner B of another model can rotate faster and uses a higher tube current to generate the same signal with a protocol of 120 kV at 400 mA for 0.75 second. As another example, a protocol for pediatric abdomen scan by scanner A indicates 80 kV, 200 mA, a helical pitch of 1, etc. Scanner B has a wider scan coverage such that a helical pitch can be translated to a single axial acquisition and uses a protocol of 70 kV, 300 mA, and axial at wide coverage. As another example, an imaging protocol for scanner A includes a priority indicating a desired limit of radiation dose level and a second priority indicating a reduction of motion artifacts by using 80 kV at 200 mA for pediatric abdomen scan. If scanner B has lower kV capabilities, the protocol for scanner B may be adjusted to 70 kV at 300 mA. As another example, scanner A has a protocol for an inner ear scan which indicates 120 kV, 200 mA, and a bone kernel filter. Scanner B has a different kernel filter that can improve bone images compared to the bone kernel filter of Scanner A but impacts the amount of signal that is required. Therefore, the impact may be accounted for such that the scanner B protocol includes 120 kV, 300 mA, and a “bone plus” kernel. Imaging procedure and associated imaging protocol(s) can be visualized via a graphical user interface (GUI) for a user (e.g., radiologist, technician, clinical specialist) to select. An interactive user interface can include menu and control options to allow the user to select and configure an imaging protocol. For an X-ray imaging protocol, the interface allows the user to select an acquisition trajectory, manage radiation dose in real-time, control tube angular orientation, tube tilt, tube position, table motion and/or orientation and other parameters for imaging during reference and/or tomosynthesis scans. When the user selects the imaging protocol via the interface, an imaging procedure associated with the imaging protocol will be performed. For an organization (e.g., hospital, clinic) that has a large fleet of imaging devices at various facilities, managing protocols for the devices can be very costly and time-consuming. Exam quality may be inconsistent due to inconsistent protocols used across the facilities, which may put patient safety and outcome at risk. Compliance with regulations and accreditation requirements may be challenging due to variability in dose, exam duration, and diagnostics quality. Protocols need to be reviewed and kept current all the time. Modification of protocols may be inefficient because protocols are modified per exam, which results in loss of productivity and revenue. An imaging protocol management system and method with improved efficiency and outcome are generally desired.

U.S. Pat. No. 9,147,041 teaches a clinical dashboard user interface method which includes the steps of displaying a navigable list of at least one target disease, displaying a navigable list of patient identifiers associated with a target disease selected in the target disease list, displaying historic and current data associated with a patient in the patient list identified as being associated with the selected target disease, including clinician notes at admission, receiving, storing, and displaying review's comments, and displaying automatically-generated intervention and treatment recommendations. One of the challenges facing hospitals today is identifying a patient's primary illness as early as possible, so that appropriate interventions can be deployed immediately. Some illnesses, such as Acute Myocardial Infarction (AMI) and pneumonia, require an immediate standard action or pathway within 24 hours of the diagnosis. Other illnesses are less acute but still require careful adherence to medium and long-term treatment plans over multiple care settings. The Joint Commission, the hospital accreditation agency approved by the Centers for Medicare and Medicaid Services (CMS), has developed Core Measures that have clearly articulated process measures. These measures are tied to standards that could result in CMS penalties for poor performance. The measures set forth for Acute Myocardial Infarction include: TABLE-US-00001 Set Measure ID # Measure Short Name AMI-1 Aspirin at Arrival AMI-2 Aspirin Prescribed at Discharge AMI-3 ACEI or ARB for LVSD AMI-4 Adult Smoking Cessation Advice/Counseling AMI-5 Beta-Blocker Prescribed at Discharge AMI-7 Median Time to Fibrionolysis AMI-7a Fibrinolytic Therapy Received within 30 minutes of Hospital Arrival AMI-8 Median Time to Primary PCI AMI-8a Primary PCI Received within 90 minutes of Hospital Arrival AMI-9 Inpatient Mortality (retired effective Dec. 31, 2010) AMI-10 Statin Prescribed at Discharge. To date, most reporting and monitoring of accountable measure activities are done after the patient has been discharged from the healthcare facility. The delay in identifying and learning about a particular intervention often makes it impossible to rectify any situation. It is also difficult for a hospital administrator to determine how well the hospital is meeting core measures daily. Clinicians need a real-time or near real-time view of patient progress and care throughout the hospital stay, including clinician notes, that will inform actions (pathways and monitoring) on the part of care management teams and physicians toward meeting these core measures. Case management teams have difficulty following patients' real-time disease status. The ability to do this with a clear picture of clinician's notes as they change in real-time as new information comes in during a patient's hospital stay would increase the teams' ability to apply focused interventions as early as possible and follow or change those pathways as needed throughout a patient's hospital stay, increasing quality and safety of care, decreasing unplanned readmissions and adverse events, and improving patient outcomes. This disclosure describes software developed to identify and risk stratify patients at highest risk for hospital readmissions and other adverse clinical events, and a dashboard user interface that presents data to the users in a clear and easy-to-understand manner.

Referring to FIG. 4 in conjunction with U.S. Pat. No. 9,147,041 a clinical predictive and monitoring system 10 includes a computer system 12 adapted to receive a variety of clinical and non-clinical data relating to patients or individuals requiring care. The variety of data include real-time data streams and historical or stored data from hospitals and healthcare entities 14, non-health care entities 15, health information exchanges 16, and social-to-health information exchanges and social services entities 17. These data are used to determine a disease risk score for selected patients so that they may receive more target intervention, treatment, and care that are better tailored and customized to their condition and needs. The clinical predictive and monitoring system 10 is most suited for identifying patients who require intensive inpatient and/or outpatient care to avert serious detrimental effects of certain diseases and to reduce hospital readmission rates. It should be noted that the computer system 12 may comprise one or more local or remote computer servers operable to transmit data and communicate via wired and wireless communication links and computer networks. The data received by the clinical predictive and monitoring system 10 may include electronic medical records (EMR) that include both clinical and non-clinical data. The EMR clinical data may be received from entities such as hospitals, clinics, pharmacies, laboratories, and health information exchanges, including: vital signs and other physiological data; data associated with comprehensive or focused history and physical exams by a physician, nurse, or allied health professional; medical history; prior allergy and adverse medical reactions; family medical history; prior surgical history; emergency room records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; physician's notes; networked devices and monitors (such as blood pressure devices and glucose meters); pharmaceutical and supplement intake information; and focused genotype testing. The EMR non-clinical data may include social, behavioral, lifestyle, and economic data; type and nature of employment; job history; medical insurance information; hospital utilization patterns; exercise information; addictive substance use; occupational chemical exposure; frequency of physician or health system contact; location and frequency of habitation changes; predictive screening health questionnaires such as the patient health questionnaire (PHQ); personality tests; census and demographic data; neighborhood environments; diet; gender; marital status; education; proximity and number of family or care-giving assistants; address; housing status; social media data; and educational level. The non-clinical patient data may further include data entered by the patients, such as data entered or uploaded to a social media website. Additional sources or devices of EMR data may provide lab results, medication assignments and changes, EKG results, radiology notes, daily weight readings, and daily blood sugar testing results. These data sources may be from different areas of the hospital, clinics, patient care facilities, patient home monitoring devices, among other available clinical or healthcare sources. Patient data sources may include non-healthcare entities 15. These are entities or organizations that are not thought of as traditional healthcare providers. These entities 15 may provide non-clinical data that include gender; marital status; education; community and religious organizational involvement; proximity and number of family or care-giving assistants; address; census tract location and census reported socioeconomic data for the tract; housing status; number of housing address changes; frequency of housing address changes; requirements for governmental living assistance; ability to make and keep medical appointments; independence on activities of daily living; hours of seeking medical assistance; location of seeking medical services; sensory impairments; cognitive impairments; mobility impairments; educational level; employment; and economic status in absolute and relative terms to the local and national distributions of income; climate data; and health registries. Such data sources may provide further insightful information about patient lifestyle, such as the number of family members, relationship status, individuals who might help care for a patient, and health and lifestyle preferences that could influence health outcomes. The clinical predictive and monitoring system 10 may further receive data from health information exchanges (HIE) 16. HIEs are organizations that mobilize healthcare information electronically across organizations within a region, community, or hospital system. HIEs are increasingly developed to share clinical and non-clinical patient data between healthcare entities within cities, states, regions, or within umbrella health systems. Data may arise from numerous sources such as hospitals, clinics, consumers, payers, physicians, labs, outpatient pharmacies, ambulatory centers, nursing homes, and state or public health agencies. A subset of HIEs connect healthcare entities to community organizations that do not specifically provide health services, such as non-governmental charitable organizations, social service agencies, and city agencies. The clinical predictive and monitoring system 10 may receive data from these social services organizations and social-to-health information exchanges 17, which may include information on daily living skills, availability of transportation to doctor appointments, employment assistance, training, substance abuse rehabilitation, counseling or detoxification, rent and utilities assistance, homeless status and receipt of services, medical follow-up, mental health services, meals and nutrition, food pantry services, housing assistance, temporary shelter, home health visits, domestic violence, appointment adherence, discharge instructions, prescriptions, medication instructions, neighborhood status, and ability to track referrals and appointments. Another source of data includes social media or social network services 18, such as FACEBOOK and GOOGLE+ websites. Such sources can provide information such as the number of family members, relationship status, identify individuals who may help care for a patient, and health and lifestyle preferences that may influence health outcomes. These social media data may be received from the websites, with the individual's permission, and some data may come directly from a user's computing device as the user enters status updates. These non-clinical patient data provides a much more realistic and accurate depiction of the patient's overall holistic healthcare environment. Augmented with such non-clinical patient data, the analysis and predictive modeling performed by the present system to identify patients at high-risk of readmission or disease recurrence become much more robust and accurate. The clinical predictive and monitoring system 10 is further adapted to receive user preference and system configuration data from clinicians' computing devices (mobile devices, tablet computers, laptop computers, desktop computers, servers, etc.) 19 in a wired or wireless manner. These computing devices are equipped to display a system dashboard and/or another graphical user interface to present system data and reports. A clinician (healthcare personnel) may immediately generate a list of patients that have the highest congestive heart failure risk scores, e.g., top n numbers or top x %. The graphical user interface is further adapted to receive the user's (healthcare personnel) input of preferences and configurations, etc. The data may be transmitted, presented, and displayed to the clinician/user in the form of web pages, web-based message, text files, video messages, multimedia messages, text messages, e-mail messages, and in a variety of suitable ways and formats.

Still referring to FIG. 4 the clinical predictive and monitoring system 10 may receive data streamed real-time, or from historic or batched data from various data sources. The clinical predictive and monitoring system 10 may store the received data in a data store 20 or process the data without storing it first. The real-time and stored data may be in a wide variety of formats according to a variety of protocols, including CCD, XDS, HL7, SSO, HTTPS, EDI, CSV, etc. The data may be encrypted or otherwise secured in a suitable manner. The data may be pulled (polled) by the clinical predictive and monitoring system 10 from the various data sources or the data may be pushed to the clinical predictive and monitoring system 10 by the data sources. The data may be received in batch processing according to a predetermined schedule or on-demand. The data store 20 may include one or more local servers, memory, drives, and other suitable storage devices. The data may be stored in a data center in the cloud. The computer system 12 may comprise several computing devices, including servers, that may be located locally or in a cloud computing farm. The data paths between the computer system 12 and the data store 20 may be encrypted or otherwise protected with security measures or transport protocols now known or later developed.

U.S. Pat. No. 10,810,787 teaches a method which includes the steps of positioning a three-dimensional imaging device within an oral cavity of a dental patient and obtaining a three-dimensional image of teeth of the dental patient using the three-dimensional imaging device and X-rays from an X-ray source external to the oral cavity and storing the three-dimensional image. The method also includes the steps of obtaining a two-dimensional image of at least one of the teeth of the dental patient using visible light from a probe-like two-dimensional imaging device inserted into the oral cavity in real time and mapping the two-dimensional image onto the stored three-dimensional image by identifying features in the two-dimensional image corresponding to features in the stored three-dimensional image using an image recognition algorithm. The probe-like two-dimensional imaging device includes a source of the visible light. The method further includes the steps of identifying the at least one of the teeth shown in the two-dimensional image in the stored three-dimensional image and displaying the stored three-dimensional image with an indication of the at least one of the teeth shown in the two-dimensional image, the indication corresponding to a current location of the probe-like two-dimensional imaging device. Methods and apparatus for obtaining images from a cavity or other difficult to reach location are known. Methods for obtaining images of a patient for use during medical procedures are known. It is useful to enable such methods and apparatus to be improved to make the images obtained easier and more informative for a medical practitioner or other user to use.

U.S. Pat. No. 10,902,595 teaches a method for populating a digital dental chart with tooth condition information for a patient's teeth which includes the steps of obtaining a digital three dimensional (3D) representation of the patient's teeth and identifying individual teeth in the digital 3D representation. The method also includes the steps of segmenting the individual teeth from the digital 3D representation and obtaining diagnostic data for one or more of the teeth. The method further includes the steps of deriving tooth condition information about specific locations of the one or more teeth from the diagnostic data, correlating the derived tooth information with the specific locations of the individual teeth, and obtaining a digital dental chart which includes regions representing surfaces of the patient's teeth. The method still further includes the steps of correlating the individual teeth with the corresponding regions of the digital dental chart and adding a representation of the derived tooth condition information to the respective specific locations at the corresponding region or regions of the digital dental chart. At least part of the diagnostic data is comprised in the digital 3D representation. The tooth condition information for a tooth is derived from variations in the diagnostic data over the segmented tooth portion of the digital 3D representation. At least part of the diagnostic data is included in a diagnostic data set obtained in addition to the digital 3D representation of the patient's teeth. The method still further includes the step of determining a spatial correlation between the digital 3D representation and the diagnostic data of the diagnostic data set. The spatial correlation between the digital 3D representation and the diagnostic data is determined by aligning corresponding portions of the digital 3D representation and the diagnostic data set. The aligning corresponding portions of the digital 3D representation and the diagnostic data is based on one or more of fiducial markers, landmark identification or aligning the surfaces using Iterative Closest Point algorithm.

The applicant hereby incorporates the above referenced patents and patent publications into their specification.

### SUMMARY OF THE INVENTION

The present invention is a method of making a diagnosis of a dental condition of a patient which includes the steps of collecting non-imaging data relating to the patient, storing the non-imaging data in a storage medium containing stored non-imaging data and existing imaging data for this patient and for a plurality of other patients and applying non-real time and non-user attended algorithms to the stored non-imaging data and existing imaging data of this patient and other patients whereby the algorithms determine the diagnosis of the dental condition of the patient.

The first aspect of the present invention is the diagnosis is complete.

The second aspect of the present invention is the diagnosis determines what new dental imaging data for the patient is required to be acquired to diagnose the dental condition of the patient.

The third aspect of the present invention is the non-imaging data includes non-clinical data and non-dental clinical data.

The fourth aspect of the present invention is the method of making a diagnosis of a dental condition of a patient includes the step of receiving diagnostic data pertaining to the patient from an oral health detection device.

The fifth aspect of the present invention is the method of making a diagnosis of a dental condition of a patient includes the steps of receiving risk factor data pertaining to the patient and processing the diagnostic data and the risk factor data on a processor to determine an oral health risk status of the patient. The step of processing the diagnostic data and the risk factor data includes determining one or more diagnostic risk measures based on the diagnostic data. At least one of the diagnostic risk measures is obtained by processing a measured diagnostic value and one or more previously measured diagnostic values for the patient.

The sixth aspect of the present invention is the method of making a diagnosis of a dental condition of a patient includes the steps of relating a rate of change of the measured diagnostic value to a risk of developing a deterioration in oral health, determining one or more patient risk measures based on the risk factor data and combining the diagnostic risk measures and the patient risk measures to obtain an integrated risk measure associated with the oral health risk status of the patient.

The seventh aspect of the present invention is the method of making a diagnosis of a dental condition of a patient includes the steps of maintaining dental, biographical, and security information for a plurality of individual patient records in a dental and biographical records database on a centralized computer, inputting patient dental and biographical information in the dental and biographical records database through a computer remotely situated from the centralized computer and inputting patient medical and biographical records security information in the medical and biographical records database through the computer remotely situated from the centralized computer. The patient dental and biographical information is information selected from the group consisting of dental history, patient genetic history, patient social history, patient mental and emotional health history, patient surgical history, patient environmental history, patient dental and oral health history, patient laboratory results, patient radiological and imaging history, patient organ system history, treatment and medication history, patient otologic and ophthalmological history, and anatomical, biochemical, physiological, pathological, and genetic histories.

The eighth aspect of the present invention is the method of making a diagnosis of a dental condition of a patient includes the steps of storing potential dental diagnoses to the patient's dental and biographical record stored on the central computer, creating a plurality of diagnostic questions relating to dental signs and symptoms requiring either a “yes” or a “no” response from a patient, storing the diagnostic questions on a central computer connected to a global computer network, differentially weighting the diagnostic questions and responses according to their relative importance in determining a dental diagnosis.

The ninth aspect of the present invention is the method of making a diagnosis of a dental condition of a patient includes the steps of retrieving patient responses to the diagnostic questions and correlating the patient responses to a list of potential diagnoses as a function of the input responses to the dental diagnostic questions and the relative weight of the dental diagnostic questions and providing the list of potential dental diagnoses to the patient via the computer network and remote computer.

The tenth aspect of the present invention is the method of making a diagnosis of a dental condition of a patient in which the non-real time and non-user attended algorithms when applied to the non-imaging data and the dental imaging data in conjunction with the stored non-imaging data and existing imaging data of this patient and other patients the non-real time and non-user attended algorithms determine what new dental imaging data for the patient is required to be acquired to diagnose a dental condition selected from a Markush Group of dental conditions including caries, stained teeth/tartar, cracked teeth, open diastema, gingivitis/periodontal disease, failing crown, failing sealant, oral candidiasis, cranial bone anomalies, tic douloureux, TMJ disorders, oral cancer or lesions, tooth erosion, unattractive smile/dimensions, dry mouth, trench mouth, bad breath, impacted tooth, implant failing/bone adherence, dry socket, atypical odontalgia, impacted wisdom tooth, dental gum or tooth abscess, mouth ulcers and bruxism of this patient.

Other aspects and many of the attendant advantages will be more readily appreciated as the same becomes better understood by reference to the following detailed description and considered in connection with the accompanying drawing in which like reference symbols designate like parts throughout the figures.

The features of the present invention which are believed to be novel are set forth with particularity in the appended claims.

### DESCRIPTION OF THE DRAWINGS

FIG. 1 is a schematic diagram of a system which diagnoses and identifies a treatment for an orthodontic condition according to U.S. Pat. No. 8,478,698 and U.S. Pat. No. 8,856,053.

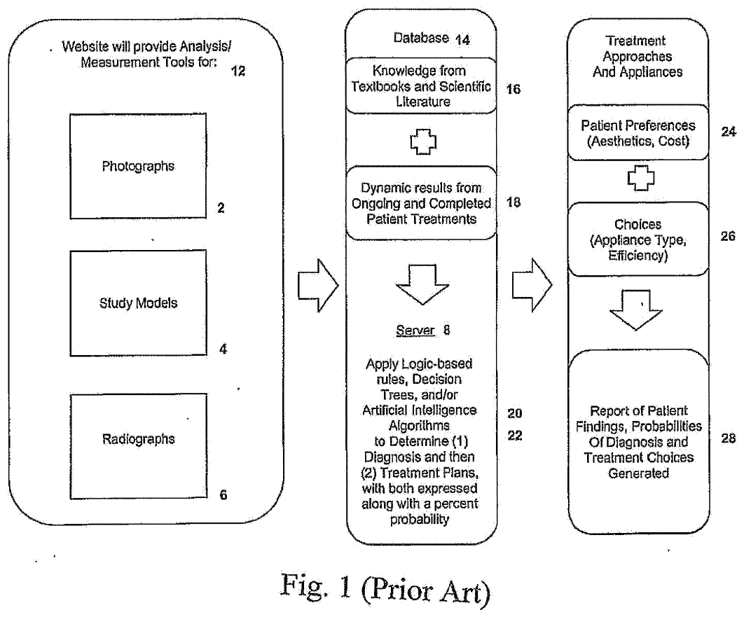

FIG. 2 is a schematic diagram of the system of FIG. 1.

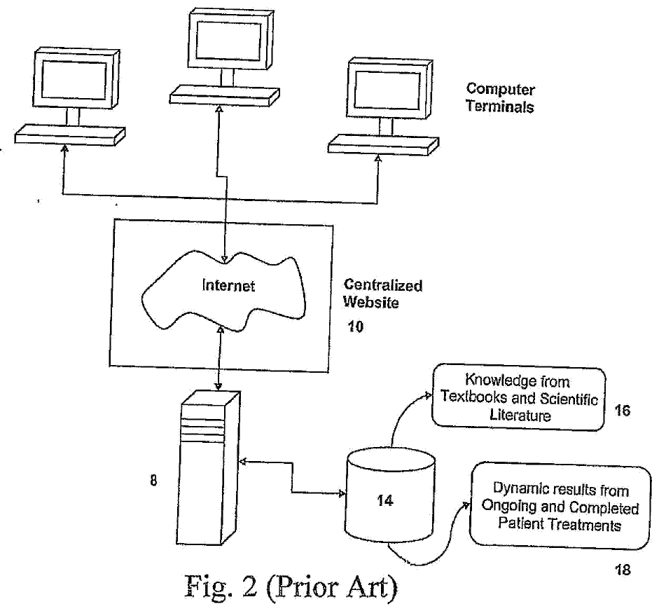

FIG. 3 is a block diagram of an automated medical and biographical and diagnostic system according to U.S. Pat. No. 7,698,154.

FIG. 4 is a simplified block diagram of a clinical predictive and monitoring system according to U.S. Pat. No. 9,147,041.

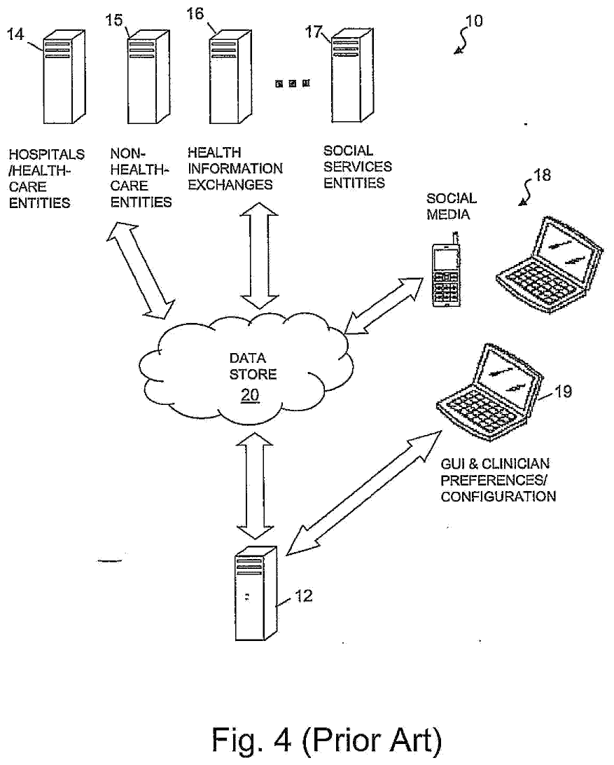

FIG. 5 is a schematic diagram of a system for a method applying non-real time and non-user attended algorithms to stored non-imaging data and existing imaging data for obtaining a dental diagnosis according to the present invention.

FIG. 6 is a flow chart of the method of FIG. 4 according to a first embodiment of the present invention.

FIG. 7 is a flow chart of the method of FIG. 4 according to a second embodiment of the present invention.

### DESCRIPTION OF THE PREFERRED EMBODIMENT

In general, the concept of the present invention is to use non-image related information from a dental practice management system to build models or statistics and then to use that to help guide the image processing which detects specific dental conditions on images. The models and statistics are built and can rely on the fact that they can house billions of images in the cloud for dentists' offices patients and can build accurate models which today is not possible because all dentists' offices images are local on their own networks. The image processing is targeted and does multiple steps and sometimes has interim detections. The algorithm might be “guided” by non-image related information that this patient has a high probability of stained teeth because the patient is a smoker. But before one can decide if a tooth is stained, he may have to detect “the gums/tissue”, and segment and find as many as the “actual teeth” as he can identify in the image (or images) and then finally he can look for “stains” in the specific teeth that were identified in the image. Many people have done image processing on teeth and other people have used other clinical associated information for some purpose such as orthodontics. Until this present invention the use of “non-real-time, unattended, multi-step image processing for dental condition detection has not yet been accomplished. The processing is at a minimum at least partially guided by using non-image related information. The method of unattended, non-real-time, dental condition detection employs automated image processing of dental images. The automated image processing is at least partially guided by non-image related dental practice management information. The software and methods are designed to detect specific dental conditions. The items detected; some of which are used as intermediaries in a multi-step analysis to reach a detectable dental condition include detection of enamel, dentine, pulp, tissue, dental enamel junction, caries, cavities, fillings, crowns, roots, periodontal ligament, implants, cracks, fissures, discoloration, stains, missing teeth, open diastema, or lesions. The algorithms use one or more non-image related information from a dental practice management system including age, nationality, sex, genetics, other medical conditions, related patients' information, non-related patients' information from the same dental office, non-related patients' information from a non-affiliated dental office, demographics, smoker status, eating habits, blood pressure, flossing habits, periodontal charting information, and previous insurance claims information. The non-image related information is used in combination with various targeted image processing operations applied to one or more physical 2D and 3D dental images of the patient. The image processing may in some cases also employ the use of statistical models and/or imaging device related knowledge to assist in guiding the image processing algorithms. Any combination of the above non-image dental practice management information can be used in combination with any of the described image processing operations and collectively is used to automatically examine in non-real time the existing stored images and volumes of a patient for detection of specific dental conditions. The image processing statistical related information and/or the practice management systems non image related information which is used for detection is preferably based upon using large depositories of dental images in combination with one or more practices non image related information which is collectively located remotely of the dental offices in off-site cloud storage and which allows automated, non-attended examination of patient dental images and information; and which software and methods can rely on vast amounts of physical images and non-image related dental practice management information from patients not only in this dental office/practice but from many related or non-related dental practices to build statistical models. The method of applying non-real time and non-user attended algorithms to stored non-imaging data and existing imaging data for obtaining a dental diagnosis relies upon access to cloud based remote storage acting as a central depository for multiple image types acquired from disparate imaging device sources and which when combined with depositories from other offices and from multiple brands of imaging devices is used to create quantifiable statistical conclusions regarding image types, image or teeth features, or common image data conditions, which can be applied to the image processing algorithms decision tree and algorithms which can partially guide the image processing algorithms and increase the ability or accuracy level of a positive detection. Dental imaging software uses the cloud for storage of 2D images and 3D volumes as central depositories for images acquired by a dental office and uses non-image related information to create statistical models which is used to partially guide the automated image processing algorithms to automatically detect without requiring user intervention during the detection. A web crawler (bot) is used to gather any possible additional associated non-image data information relative to this patient, groups of patients, conditions or groups of conditions, current studies or articles, newly emerging techniques or information regarding this dental condition or patient or group of patients, or any known medical procedures that have been performed on this patient or related patient; which any or all can be used to help guide the decision tree in the detection algorithms. The method greatly reduces the amount of time required for a dentist to screen for dental conditions by employing software algorithms (automated and/or web crawler software) which use a combination of statistical or probabilistic information; x-ray information; and dentin and tissue related information and which non image related information is used to partially assist in guiding the detection algorithms. The method helps improve the issue of under diagnosis of dental conditions in the dental practice by providing an unattended, non-real-time, automated dental conditions detection method and software using automated image processing of dental 2D images or 3D volumes, and which automated image processing is guided by non-image related dental practice management system information.

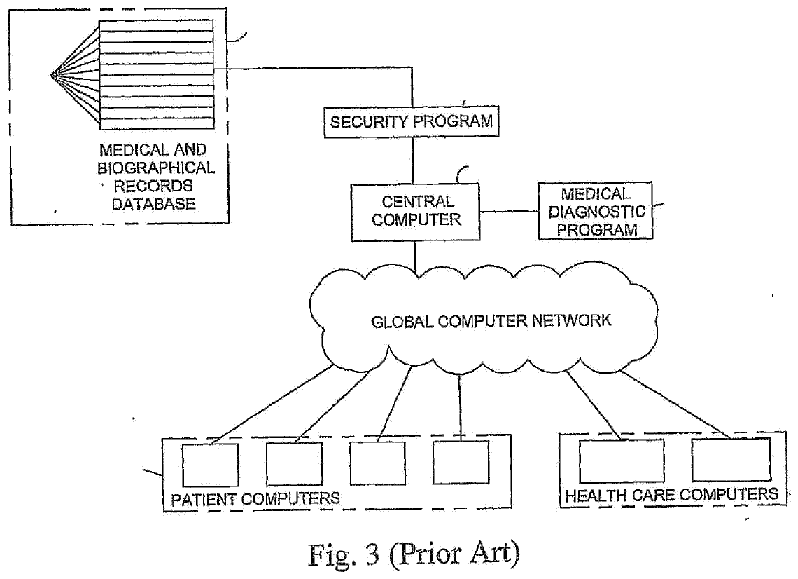

Again, referring to FIG. 3 is a schematic diagram of an automated medical and biographical and diagnostic system in which an individual patient's medical and biographical record information can be accessed, added, modified, maintained, and controlled by the patient. The automated medical and biographical and diagnostic system provides medical diagnostic information in which the patient obtains a list of potential medical diagnoses corresponding to input health symptoms. The automated medical and biographical and diagnostic system includes a central computer that is connected to a global computer network. The central computer has access to a medical and biographical records database that contains a plurality of medical and biographical records for individual patients. Connected to global computer network are a plurality of patient computers and health care computers. Patients obtain access to their medical and biographical records by accessing central computer via patient computers connected to global computer network. The central computer executes security program that limits access to medical and biographical database and individual medical and biographical records contained therein. Once a patient's identity is verified by security program, the patient may gain access to his or her own individual medical and biographical record. Health care providers obtain access to patients medical and biographical records by accessing central computer via health care computers connected to global computer network. The central computer executes security program to limit access to medical and biographical database and individual medical and biographical records contained therein to health care providers that are authorized by a patient to access a medical and biographical record of a patient. The creation and maintenance of medical records, includes the steps of recording and correlating past medical history and biographical information, integrating genetic, laboratory, radiologic, and imaging results, prescribed medications, and treatments, noting patient allergies, reactions, and treatment outcome and updating medical records

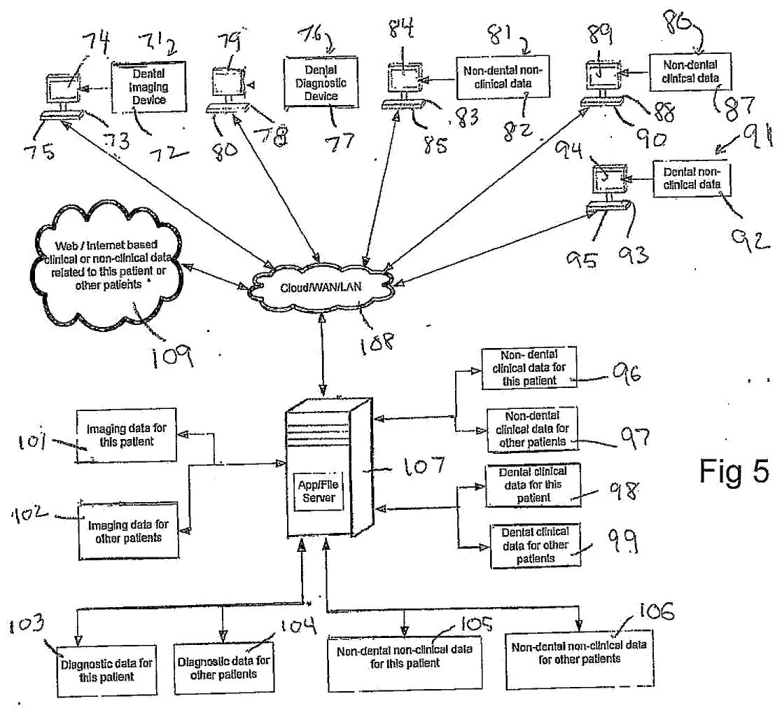

Referring to FIG. 5 in conjunction with FIG. 3 a dental diagnostic system 70 uses a method that applies non-real time and non-user attended algorithms to stored non-imaging data and existing imaging data for obtaining a dental diagnosis. The dental diagnostic system 70 has a dental imaging module 71 which includes a dental imaging device 72 and a first computer 73 with a microprocessor, a display 74, a keyboard 75 and a memory and a dental diagnostic module 76 which includes a dental diagnostic device 77 and a second computer 78 with a microprocessor, a display 79, a keyboard 80 and a memory. The dental diagnostic system 70 also has a non-dental, non-clinical data module 81 which includes a non-dental, non-clinical data source 82 and a third computer 83 with a microprocessor, a display 84, a keyboard 85 and a memory, a non-dental clinical data module 86 which includes a non-dental clinical data source 87 and a fourth computer 88 with a microprocessor, a display 89, a keyboard 90 and a memory and a dental non-clinical data module 91 which includes a dental non-clinical data source 92 and a fifth computer 93 with a microprocessor, a display 94, a keyboard 95 and a memory. The dental diagnostic system 70 further has a first source 96 of non-dental clinical data for this patient, a second source 97 of non-dental clinical data for other patients, a third source 98 for dental clinical data for this patient and a fourth source 99 of dental clinical data for other patients. The dental diagnostic system 70 still further has a fifth source 101 of imaging data for this patient, a sixth source 102 of imaging data for other patients, a seventh source 103 for diagnostic data for this patient, an eight source 104 of diagnostic data for other patients, a ninth source 105 of non-dental non-clinical data for this patient and a tenth source 106 of non-dental non-clinical data for other patients. The dental diagnostic system 70 further still has a server 107 which contains software, applications and algorithms for providing a dental diagnosis and which is coupled to the Cloud/WAN/LAN 108. There may also be a web/internet-based source 109 of clinical or non-clinical data related to this patient or other patients. Each of the dental imaging module 71, the dental diagnostic module 76, the non-dental, non-clinical data module 81, the non-dental clinical data module 86 and the dental non-clinical data module 91 is interactively coupled to the software, applications, and algorithms of the server 107. Each of the first source 96 of non-dental clinical data for this patient, the second source 97 of non-dental clinical data for other patients, the third source 98 for dental clinical data for this patient, the fourth source 99 of dental clinical data for other patients, the fifth source 101 of imaging data for this patient, the sixth source 102 of imaging data for other patients, the seventh source 103 for diagnostic data for this patient, the eight source 104 of diagnostic data for other patients, the ninth source 105 of non-dental non-clinical data for this patient and the tenth source 106 of non-dental non-clinical data for other patients is interactively coupled to the software, applications and algorithms of the server 107.

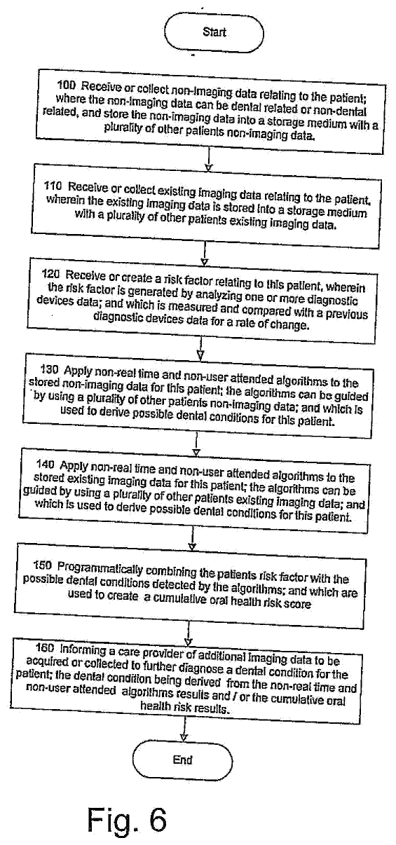

Referring to FIG. 6 in conjunction with FIG. 5 in Step 100 the modules either receive or collect non-imaging data relating to the patient. The non-imaging data can be either dental related or non-dental related. The modules store the non-imaging data into a storage medium with a plurality of other patients' non-imaging data. In Step 110 the modules either receive or collect existing imaging data relating to the patient. The existing imaging data is stored into a storage medium with a plurality of other patients' existing imaging data. In Step 120 the modules either receive or create a risk factor relating to this patient. The risk factor is generated by analyzing data from one or more diagnostic device. The data is measured and compared with data of previously used diagnostic devices for a rate of change. In Step 130 the server 107 applies non-real time and non-user attended algorithms to the stored non-imaging data for this patient. The algorithms can be guided by using a plurality of other patients' non-imaging data and is used to derive possible dental conditions for this patient. In Step 140 the server 107 applies non-real time and non-user attended algorithms to the stored existing imaging data for this patient. The algorithms can be guided by using a plurality of other patients' existing imaging data which is used to derive possible dental conditions for this patient. In Step 150 the server 107 programmatically combines the patient's risk factor with the possible dental conditions detected by the algorithms which are used to create a cumulative oral health risk score. In Step 160 the dental diagnostic system 70 informs a care provider of additional imaging data to be acquired or collected to further diagnose a dental condition for this patient. The dental condition is derived from the non-real time and non-user attended algorithms results and/or the cumulative oral health risk results.

Referring to FIG. 6 a first method of making a diagnosis of a dental condition of a patient includes the steps of collecting non-imaging data relating to the patient, storing the non-imaging data in a storage medium containing stored non-imaging data and existing imaging data for this patient and for a plurality of other patients and applying non-real time and non-user attended algorithms to the stored non-imaging data and existing imaging data of this patient and other patients. The algorithms determine the diagnosis of the dental condition of the patient. The diagnosis is either a complete diagnosis or determination of what new dental imaging data for the patient is required to be acquired to diagnose the dental condition of the patient. The non-imaging data includes non-clinical data and non-dental clinical data.

Still referring to FIG. 6 the first method of making a diagnosis of a dental condition of a patient also includes the steps of receiving diagnostic data pertaining to the patient from an oral health detection device, receiving risk factor data pertaining to the patient, processing the diagnostic data and the risk factor data on a processor to determine an oral health risk status of the patient. The step of processing the diagnostic data and the risk factor data includes determining one or more diagnostic risk measures based on the diagnostic data. At least one of the diagnostic risk measures is obtained by processing a measured diagnostic value and one or more previously measured diagnostic values for the patient and relating a rate of change of the measured diagnostic value to a risk of developing deterioration in oral health. The first method of making a diagnosis of a dental condition of a patient further includes the steps of determining one or more patient risk measures based on the risk factor data and combining the diagnostic risk measures and the patient risk measures to obtain an integrated risk measure associated with the oral health risk status of the patient. The first method of making a diagnosis of a dental condition of a patient still further includes the steps of maintaining dental, biographical and security information for a plurality of individual patient records in a dental and biographical records database on a centralized computer, inputting patient dental and biographical information in the dental and biographical records database through a computer remotely situated from the centralized computer and inputting patient medical and biographical records security information in the medical and biographical records database through the computer remotely situated from the centralized computer. The patient dental and biographical information is information selected from the group consisting of dental history, patient genetic history, patient social history, patient mental and emotional health history, patient surgical history, patient environmental history, patient dental and oral health history, patient laboratory results, patient radiological and imaging history, patient organ system history, treatment and medication history, patient otologic and ophthalmological history, and anatomical, biochemical, physiological, pathological, and genetic histories. The first method of making a diagnosis of a dental condition of a patient further still includes the steps of storing potential dental diagnoses to the patient's dental and biographical record stored on the central computer, creating a plurality of diagnostic questions relating to dental signs and symptoms requiring either a “yes” or a “no” response from a patient, storing the diagnostic questions on a central computer connected to a global computer network and differentially weighting the diagnostic questions and responses according to their relative importance in determining a dental diagnosis, providing a software program interface accessible by computers situated remotely from the central computer. The interface interactively displays to patients a series of the diagnostic questions stored on the central computer. The first method of making a diagnosis of a dental condition of a patient also still further includes the steps of retrieving patient responses to the diagnostic questions and correlating the patient responses to a list of potential diagnoses as a function of the input responses to the dental diagnostic questions and the relative weight of the dental diagnostic questions and providing the list of potential dental diagnoses to the patient via the computer network and remote computer. By processing a measured diagnostic value and one or more previously measured diagnostic values for the patient and relating a rate of change of the measured diagnostic value to a risk of developing deterioration in oral health. The first method of making a diagnosis of a dental condition of a patient still also further includes the steps of determining one or more patient risk measures based on the risk factor data and combining the diagnostic risk measures and the patient risk measures to obtain an integrated risk measure associated with the oral health risk status of the patient.

Still referring to FIG. 5 in conjunction with FIG. 6 a method of making a diagnosis of a dental condition of a patient includes the steps of collecting non-imaging data relating to the patient, storing the non-imaging data in a storage medium containing stored non-imaging data and existing imaging data for this patient and for a plurality of other patients, and collecting current imaging data for the patient. The method also includes the steps of transferring the existing and current imaging data to a central processing unit so that the central processing unit has access to a database having information associated with dental conditions, applying non-real time and non-user attended algorithms to the stored non-imaging data and the existing and current imaging data of this patient and other patients whereby the algorithms determine the diagnosis of a dental condition of the patient. The method also includes the steps of predicting orthodontic conditions of the patient based upon the measurements and the information in the database and recommending treatments to the patient based upon the predicted dental conditions. The central processing unit provides predictions based on the existing and current imaging data and the information in the database. The central processing unit also provides recommendations based on the existing and current imaging data and the information in the database.

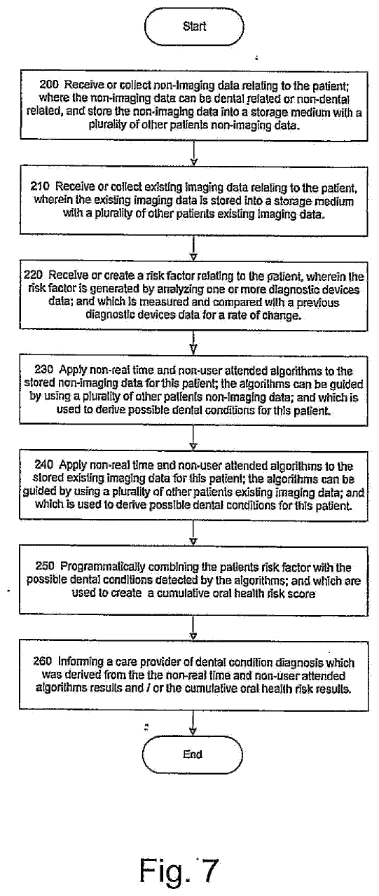

Referring to FIG. 7 in conjunction with FIG. 5 in Step 200 the modules either receive or collect non-imaging data relating to the patient. The non-imaging data can be either dental related or non-dental related. The modules store the non-imaging data into a storage medium with a plurality of other patients' non-imaging data. In Step 210 the modules either receive or collect existing imaging data relating to the patient. The existing imaging data is stored into a storage medium with a plurality of other patients' existing imaging data. In Step 220 the modules either receive or create a risk factor relating to this patient. The risk factor is generated by analyzing data from one or more diagnostic device. The data is measured and compared with data of previously used diagnostic devices for a rate of change. In Step 230 the server 107 applies non-real time and non-user attended algorithms to the stored non-imaging data for this patient. The algorithms can be guided by using a plurality of other patients' non-imaging data and is used to derive possible dental conditions for this patient. In Step 240 the server 107 applies non-real time and non-user attended algorithms to the stored existing imaging data for this patient. The algorithms can be guided by using a plurality of other patients' existing imaging data which is used to derive possible dental conditions for this patient. In Step 250 the server 107 programmatically combines the patient's risk factor with the possible dental conditions detected by the algorithms which are used to create a cumulative oral health risk score. In Step 260 the dental diagnostic system 70 informs a care provider of a dental condition diagnosis which was derived from the non-real time and non-user attended algorithms results and/or the cumulative oral health risk results.

Referring to FIG. 7 a second method of method of making a diagnosis of a dental condition of a patient includes the steps of collecting non-imaging data relating to the patient, collecting dental imaging data relating to the patient, storing the non-imaging data and the dental imaging data in a storage medium containing stored non-imaging data and existing imaging data for this patient and a plurality of other patients and applying non-real time and non-user attended algorithms to the stored non-imaging data and existing imaging data of this patient and other patients. The algorithms diagnose the dental condition of the patient. The non-imaging data includes non-clinical data and non-dental clinical data.

Still referring to FIG. 7 the second method of making a diagnosis of a dental condition of a patient also includes the steps of receiving diagnostic data pertaining to the patient from an oral health detection device, receiving risk factor data pertaining to the patient, processing the diagnostic data and the risk factor data on a processor to determine an oral health risk status of the patient. The step of processing the diagnostic data and the risk factor data includes determining one or more diagnostic risk measures based on the diagnostic data. At least one of the diagnostic risk measures is obtained by processing a measured diagnostic value and one or more previously measured diagnostic values for the patient and relating a rate of change of the measured diagnostic value to a risk of developing deterioration in oral health. The first method of making a diagnosis of a dental condition of a patient further includes the steps of determining one or more patient risk measures based on the risk factor data and combining the diagnostic risk measures and the patient risk measures to obtain an integrated risk measure associated with the oral health risk status of the patient. The second method of making a diagnosis of a dental condition of a patient still further includes the steps of maintaining dental, biographical and security information for a plurality of individual patient records in a dental and biographical records database on a centralized computer, inputting patient dental and biographical information in the dental and biographical records database through a computer remotely situated from the centralized computer and inputting patient medical and biographical records security information in the medical and biographical records database through the computer remotely situated from the centralized computer. The patient dental and biographical information is information selected from the group consisting of dental history, patient genetic history, patient social history, patient mental and emotional health history, patient surgical history, patient environmental history, patient dental and oral health history, patient laboratory results, patient radiological and imaging history, patient organ system history, treatment and medication history, patient otological and ophthalmological history, and anatomical, biochemical, physiological, pathological, and genetic histories. The second method of making a diagnosis of a dental condition of a patient further still includes the steps of storing potential dental diagnoses to the patient's dental and biographical record stored on the central computer, creating a plurality of diagnostic questions relating to dental signs and symptoms requiring either a “yes” or a “no” response from a patient, storing the diagnostic questions on a central computer connected to a global computer network and differentially weighting the diagnostic questions and responses according to their relative importance in determining a dental diagnosis, providing a software program interface accessible by computers situated remotely from the central computer. The interface interactively displays to patients a series of the diagnostic questions stored on the central computer. The second method of making a diagnosis of a dental condition of a patient also still further includes the steps of retrieving patient responses to the diagnostic questions and correlating the patient responses to a list of potential diagnoses as a function of the input responses to the dental diagnostic questions and the relative weight of the dental diagnostic questions and providing the list of potential dental diagnoses to the patient via the computer network and remote computer. By processing a measured diagnostic value and one or more previously measured diagnostic values for the patient and relating a rate of change of the measured diagnostic value to a risk of developing deterioration in oral health. The second method of making a diagnosis of a dental condition of a patient still also further includes the steps of determining one or more patient risk measures based on the risk factor data and combining the diagnostic risk measures and the patient risk measures to obtain an integrated risk measure associated with the oral health risk status of the patient.

Both embodiments of the methods of making a diagnosis of a dental condition of a patient use non-real time and non-user attended algorithms. When these algorithms are applied to non-imaging data and dental imaging data in conjunction with stored non-imaging data and existing imaging data of this patient and other patients the non-real time and non-user attended algorithms determine what new dental imaging data for the patient is required to be acquired to diagnose a dental condition selected from a Markush Group of dental conditions including caries, stained teeth/tartar, cracked teeth, open diastema, gingivitis/periodontal disease, failing crown, failing sealant, oral candidiasis, cranial bone anomalies, tic douloureux, TMJ disorders, oral cancer or lesions, tooth erosion, unattractive smile/dimensions, dry mouth, trench mouth, bad breath, impacted tooth, implant failing/bone adherence, dry socket, atypical odontalgia, impacted wisdom tooth, dental gum or tooth abscess, mouth ulcers and bruxism of this patient.

The images, measurements and/or landmarks from the IC may be taken by the wand and may be processed by the CPU. Information otherwise gathered by the system may be processed by the CPU. The CPU may have a data output component (hereinafter referred to as “DOC”). The information, images, measurements and/or landmarks may be transmitted, electronically or otherwise, by the DOC. The DOC may transmit images and/or data to another location via the internet, electronic mail, or other means, for evaluation by another system or individual, such as a doctor, dentist, orthodontist, or the like. The DOC may be implemented by one skilled in the art such that the DOC may transmit images and/or data by the internet, telephony, satellite, or other means. The DOC may generate a document for the patient. The IC and/or the wand may transmit digital and/or analog signals that may represent the images of the mouth and/or the dentition of the patient to the CPU. The CPU may perform calculations and/or a diagnosis based upon the images, preprogrammed information and/or any other information that may be entered by the user. After the diagnosis may be complete, the CPU may instruct the patient about treatments for the specific orthodontic conditions. The database may be connected to the CPU of the system. The database may store information regarding medical, orthodontic and/or dental conditions, growth charts, multiplication factors for estimations, standardized measurements and/or the like. Sizes of dentition for patients of various age ranges may be stored in the database.

From the foregoing, methods of making a diagnosis of a dental condition of a patient by applying non-real time and non-user non-imaging data and existing imaging data to obtain a dental diagnosis have been described.

Accordingly, it is intended that the foregoing disclosure and showing made in the drawing shall be considered only as an illustration of the principle of the present invention.

What is claimed is:

1. A method of making a diagnosis of a dental condition of a patient comprising the steps of:
a. collecting non-imaging data relating to the patient;
b. storing said non-imaging data in a storage medium containing stored non-imaging data and existing imaging data for this patient and for a plurality of other patients;
c. collecting current imaging data for the patient'
d. transferring said existing and current imaging data to a central processing unit wherein said central processing unit has access to a database having information associated with dental conditions; and
c. applying non-real time and non-user attended algorithms to said stored non-imaging data and said existing and current imaging data of this patient and other patients whereby said algorithms determine the diagnosis of a dental condition of the patient.

2. A method of making a diagnosis of a dental condition of a patient according to claim 1 wherein the diagnosis is complete.

3. A method of making a diagnosis of a dental condition of a patient according to claim 1 wherein the diagnosis determines what new dental imaging data for the patient is required to be acquired to diagnose the dental condition of the patient.

4. A method of making a diagnosis of a dental condition of a patient according to claim 1 wherein said non-imaging data includes non-clinical data and non-dental clinical data.

5. A method of making a diagnosis of a dental condition of a patient according to claim 2 including the steps of:
a. receiving diagnostic data pertaining to the patient from an oral health detection device;
b. receiving risk factor data pertaining to the patient; processing said diagnostic data and said risk factor data on a processor to determine an oral health risk status of the patient wherein said step of processing said diagnostic data and said risk factor data includes determining one or more diagnostic risk measures based on said diagnostic data, wherein at least one of said diagnostic risk measures is obtained by processing a measured diagnostic value and one or more previously measured diagnostic values for the patient, and relating a rate of change of said measured diagnostic value to a risk of developing a deterioration in oral health; and
c. determining one or more patient risk measures based on said risk factor data; and d. combining said diagnostic risk measures and said patient risk measures to obtain an integrated risk measure associated with said oral health risk status of the patient.

6. A method of making a diagnosis of a dental condition of a patient according to claim 5 including the steps of:
a. maintaining dental, biographical, and security information for a plurality of individual patient records in a dental and biographical records database on a centralized computer;
b. inputting patient dental and biographical information in the dental and biographical records database through a computer remotely situated from the centralized computer;
c. inputting patient medical and biographical records security information in the medical and biographical records database through the computer remotely situated from the centralized computer wherein the patient dental and biographical information is information selected from the group consisting of dental history, patient genetic history, patient social history, patient mental and emotional health history, patient surgical history, patient environmental history, patient dental and oral health history, patient laboratory results, patient radiological and imaging history, patient organ system history, treatment and medication history, patient otologic and ophthalmological history, and anatomical, biochemical, physiological, pathological, and genetic histories;
d. storing potential dental diagnoses to the patient's dental and biographical record stored on the central computer;
e. creating a plurality of diagnostic questions relating to dental signs and symptoms requiring either a “yes” or a “no” response from a patient;
f. storing said diagnostic questions on a central computer connected to a global computer network;
g. differentially weighting the diagnostic questions and responses according to their relative importance in determining a dental diagnosis;
h. providing a software program interface accessible by computers situated remotely from the central computer wherein said interface interactively displays to patients a series of the diagnostic questions stored on the central computer;
i. retrieving patient responses to the diagnostic questions and correlating the patient responses to a list of potential diagnoses as a function of the input responses to the dental diagnostic questions and the relative weight of the dental diagnostic questions; and
j. providing the list of potential dental diagnoses to the patient via the computer network and remote computer.

7. A method of making a diagnosis of a dental condition of a patient comprising the steps of:
a. collecting non-imaging data relating to the patient; b. collecting dental imaging data relating to the patient;
c. storing said non-imaging data and said dental imaging data in a storage medium containing stored non-imaging data and existing imaging data for this patient and a plurality of other patients; and
d. applying non-real time and non-user attended algorithms to said stored non-imaging data and existing imaging data of this patient and other patients whereby said algorithms diagnose the dental condition of the patient.

8. A method of making a diagnosis of a dental condition of a patient according to claim 7 wherein said non-imaging data includes non-clinical data and non-dental clinical data.

9. A method of making a diagnosis of a dental condition of a patient according to claim 7 including the steps of: a. receiving diagnostic data pertaining to the patient from an oral health detection device; b. receiving risk factor data pertaining to the patient; processing said diagnostic data and said risk factor data on a processor to determine an oral health risk status of the patient wherein said step of processing said diagnostic data and said risk factor data includes determining one or more diagnostic risk measures based on said diagnostic data, wherein at least one of said diagnostic risk measures is obtained by processing a measured diagnostic value and one or more previously measured diagnostic values for the patient, and relating a rate of change of said measured diagnostic value to a risk of developing a deterioration in oral health; c. determining one or more patient risk measures based on said risk factor data; and d. combining said diagnostic risk measures and said patient risk measures to obtain an integrated risk measure associated with said oral health risk status of the patient.

10. A method of making a diagnosis of a dental condition of a patient according to claim 9 including the steps of:
a. maintaining dental, biographical, and security information for a plurality of individual patient records in a dental and biographical records database on a centralized computer;
b. inputting patient dental and biographical information in the dental and biographical records database through a computer remotely situated from the centralized computer;
c. inputting patient medical and biographical records security information in the medical and biographical records database through the computer remotely situated from the centralized computer wherein the patient dental and biographical information is information selected from the group consisting of dental history, patient genetic history, patient social history, patient mental and emotional health history, patient surgical history, patient environmental history, patient dental and oral health history, patient laboratory results, patient radiological and imaging history, patient organ system history, treatment and medication history, patient otologic and ophthalmological history, and anatomical, biochemical, physiological, pathological, and genetic histories;
d. storing potential dental diagnoses to the patient's dental and biographical record stored on the central computer;
e. creating a plurality of diagnostic questions relating to dental signs and symptoms requiring either a “yes” or a “no” response from a patient;
f. storing the diagnostic questions on a central computer connected to a global computer network;
g. differentially weighting the diagnostic questions and responses according to their relative importance in determining a dental diagnosis;
h. providing a software program interface accessible by computers situated remotely from the central computer wherein the interface interactively displays to patients a series of the diagnostic questions stored on the central computer;
i. retrieving patient responses to the diagnostic questions and correlating the patient responses to a list of potential diagnoses as a function of the input responses to the dental diagnostic questions and the relative weight of the dental diagnostic questions; and
j. providing the list of potential dental diagnoses to the patient via the computer network and remote computer.

11. A method of making a diagnosis of a dental condition of a patient comprising the steps of:
a. using an oral health detection device to collect non-imaging data relating to the patient;
b. storing the non-imaging data into a storage medium containing stored non-imaging data and existing imaging data for this patient and a plurality of other patients; and
c. using the oral health detection device to apply non-real time and non-user attended algorithms to the non-imaging data in conjunction with the stored non-imaging data and existing imaging data of this patient and the other patients whereby the algorithms determine what new dental imaging data for the patient is required to be acquired to diagnose the dental condition of the patient.

12. A method of making a diagnosis of a dental condition of a patient according to claim 11 wherein the non-imaging data includes not only non-clinical data and non-dental clinical data, but also risk factors;

13. A method of making a diagnosis of a dental condition of a patient according to claim 11 including the steps of:
a. receiving the diagnostic data pertaining to the patient from an oral health detection device;
b. receiving the risk factor data pertaining to the patient; processing the diagnostic data and the risk factor data on a processor to determine an oral health risk status of the patient wherein the step of processing the diagnostic data and the risk factor data includes determining one or more diagnostic risk measures based on the diagnostic data, wherein at least one of the diagnostic risk measures is obtained by processing a measured diagnostic value and one or more previously measured diagnostic values for the patient, and relating a rate of change of the measured diagnostic value to a risk of developing a deterioration in oral health;
c. determining one or more patient risk measures based on the risk factor data; and
d. combining the diagnostic risk measures and the patient risk measures to obtain an integrated risk measure associated with the oral health risk status of the patient.

14. A method of making a diagnosis of a dental condition of a patient according to claim 4 wherein the non-real time and non-user attended algorithms when applied to the non-imaging data and the dental imaging data in conjunction with the stored non-imaging data and existing imaging data of this patient and other patients the non-real time and non-user attended algorithms determine what new dental imaging data for the patient is required to be acquired to diagnose a dental condition selected from a Markush Group of caries, stained teeth/tartar, cracked teeth, open diastema, gingivitis/periodontal disease, failing crown, failing sealant, oral candidiasis, cranial bone anomalies, tic douloureux, TMJ disorders, oral cancer or lesions, tooth erosion, unattractive smile/dimensions, dry mouth, trench mouth, bad breath, impacted tooth, implant failing/bone adherence, dry socket, atypical odontalgia, impacted wisdom tooth, dental gum or tooth abscess, mouth ulcers and bruxism of this patient.

15. A method of making a diagnosis of a dental condition of a patient according to claim 9 wherein the non-real time and non-user attended algorithms when applied to the stored non-imaging data and existing imaging data of this patient and other patients wherein the non-real time and non-user attended algorithms diagnose a dental condition selected from a Markush Group of caries, stained teeth/tartar, cracked teeth, open diastema, gingivitis/periodontal disease, failing crown, failing sealant, oral candidiasis, cranial bone anomalies, tic douloureux, TMJ disorders, oral cancer or lesions, tooth erosion, unattractive smile/dimensions, dry mouth, trench mouth, bad breath, impacted tooth, implant failing/bone adherence, dry socket, atypical odontalgia, impacted wisdom tooth, dental gum or tooth abscess, mouth ulcers and bruxism of this patient.

16. A method of making a diagnosis of a dental condition of a patient according to claim 1 wherein said method also includes the steps of:
a. predicting orthodontic conditions of the patient based upon the measurements and the information in the database wherein said central processing unit provides predictions based on the imaging data and the information in said database; and
b. recommending treatments to the patient based upon the predicted dental orthodontic conditions wherein said central processing unit provides recommendations based on said existing and current imaging data and the information in said database.
